(19) United States
(12) Patent Application Publication
(10) Pub. No.: US 2005/0019855 A1
(43) Pub. Date: Jan. 27, 2005

(54) Methods to increase the productivity of meterologous gene expression

(76) Inventor: Jurgen Denecke, Heworth (DE)

Correspondence Address: MYERS BIGEL SIBLEY & SAJOVEC, PO BOX 37428, RALEIGH, NC, 27627, US

(21) Appl. No.: 10/489,483

(22) PCT Filed: Sep. 2, 2002
(86) PCT No.: PCT/GB02/04003

(30) Foreign Application Priority Data
Aug. 31, 2001 (GB) 012108 8

(51) Int. Cl. C12P 21/02; C07K 14/565

(52) U.S. Cl. 435/69.1; 435/320.1; 435/325; 530/399; 536/23.5

(57) ABSTRACT

The present invention provides a method of increasing heterologous protein expression/production in a cell by controlling proteolysis in the post ER compartment of the cell Proteolysis can be controlled by limiting/preventing export of proteins from the ER to the post ER compartment of the cell and/or by re-directing proteins from the vacuolar sorting route back to the ER or on towards the cell surface.

The present invention relates to a method of increasing heterologous protein production in cells, cells which have been adapted to increase heterologous protein production and uses of such cells.

### BACKGROUND TO THE INVENTION

Protein synthesis and secretion by the secretory pathway occurs at the endoplasmic reticulum (ER) which maintains high levels of soluble residents such as the lumenal binding protein (BiP), protein disulfide isomerase or calreticulin. The concentration of non-residents in the ER lumen which are in transit to other compartments, such as vacuoles or the extracellular matrix, is usually much lower. Despite this, export of non-resident proteins from the ER is efficient while the cells are able to restrict the leakage of the far more abundant ER residents to a minimum. In spite of significant advances in our understanding of the mechanisms underlying vesicle budding and transport between the ER and the Golgi, considerably less is known about the sorting of soluble cargo molecules during ER export.

The first model for protein secretion was inspired by the fact that different secreted proteins are secreted at various rates which implied that active export signals with different affinities for an ER export receptor exist. An alternative model was later proposed in which sorting signals were envisaged to have a role as retention signals to deviate proteins from a default route that leads to secretion (Wieland et al., 1987). More recently (Barlowe et al., 1994) it has been observed that COPII vesicles, which carry out anterograde transport between the ER and the Golgi, were found to be enriched for secretory cargo but lacked the ER resident protein BiP. This observation indicated that secretory cargo is selected and concentrated into COPII vesicles during budding so that ER residents are excluded. Further evidence for the concentration of secretory cargo during export was provided by the identification of a di-acidic ER export signal (Asp-X-Glu, where X is any amino acid) in the cytosolic tail of vesicular stomatitis glycoprotein (Balch et al., 1994; Nishimura and Balch, 1997).

These results suggest the presence of an export receptor. However, identification of an export signal on any soluble protein remains elusive to date.

Eukaryotic cells of fungi, yeasts, algae, plants, insects and mammals all share the same secretory pathway consisting of the endoplasmic reticulum, the Golgi apparatus and lytic (vacuolar) compartments. Plant cells contain at least two functionally distinct vacuolar compartments, the central lytic vacuole which is related to the mammalian lysosome, and the so-called storage vacuoles, which appear to be unique to the plant kingdom. Whereas the lytic vacuole is typical for vegetative cells, storage vacuoles are mostly found in reserve tissues of seeds. Exceptions to this are the vegetative storage vacuoles which are formed during stress conditions and which could be related to a neutral vacuolar compartment recently discovered in tobacco protoplasts. It was initially thought that the various vacuolar compartments share a common origin but change appearance and contents according to the physiological conditions or tissue type. However, the simultaneous presence of storage vacuoles and lytic vacuoles within the same cell, as determined using specific membrane markers, argues against this. In addition, the great variety of vacuolar sorting signals described in plants also points at the possibility that the various types of vacuoles have a different origin and are supported by different protein transport pathways.

It is apparent that there are many factors involved in ER export and vacuolar sorting which are are yet to be identified and which may reveal further unique features of the plant secretory pathway. To date, a single putative vacuolar sorting receptor has been identified in plants (BP80 or VSR_{PS-1}) and tested either by in vitro ligand binding assays (Kirsch et al., 1994) or via re-constitution in a heterologous in vivo system using yeast (Humair et al., 2001).

It is believed that disposal of proteins may be as a result of a “quality control” mechanism in the secretory pathway, which may recognise heterologous proteins as unsuitable for secretion and signals targeting to the possible disposal sites, which comprise the lytic vacuole or the cytosolic proteasome. The present invention provides a method to purposefully prevent such disposal from occurring.

Heterologous protein production using the secretory pathway of plants and/or micro-organisms is a potentially commercially valuable method of producing, for example, mammalian serum proteins. However a problem with such a method is that the secretion of heterologous proteins is often low-yielding which has thus prevented heterologous protein production by the plant secretory pathway to enter into widespread industrial use/applications.

Attempts have been made to increase the yield of proteins produced by the secretory pathway, which have been disappointing. Prior art methods have attempted to solve the problem of low yields by increasing the rate of synthesis. The solution provided by the present invention is thus innovative compared to the teachings in the prior art.

A method which could improve on prior art performance and increase the yield of heterologous protein production in plant and/or microbial cells would offer immediate advantage to the art.

Moreover, such a method would be applicable to the large scale production of many mammalian proteins and other proteins of high value.

### STATEMENT OF THE INVENTION

According to the broadest aspect of the invention there is provided a method of increasing heterologous protein expression/production by cell by regulating/controlling proteolysis, especially in the post ER compartment of the cell.

The present invention is based on the unexpected observation that proteolysis, and not synthesis, is a limiting factor in heterologous protein production.

Reference herein to heterologous protein is intended to include any foreign or non-native protein that is produced by the cell.

According to an aspect of the invention there is provided a method of increasing heterologous protein production in a cell comprising limiting and/or decreasing proteolysis by:
(i) limiting/preventing export of proteins from the ER to the post ER compartment of the cell;
(ii) re-directing proteins from the vacuolar sorting route back to the ER; and/or
(iii) re-directing proteins from the vacuolar sorting route to the cell surface.

For strategy (i), the host cell can be of any eukaryotic origin, i.e. plant, fungal, yeast or mammalian origin. For strategies (ii) and (iii), the method is restrictive to plants but may be applied to other eukaryotic cells with or without minor modifications.

According to a further aspect of the invention there is provided a cell adapted so that proteolysis is limited and/or decreased.

Preferably the cell is adapted by the method of the present invention.

According to a yet further aspect of the invention there is provided use of the cell/cells of the present invention in increasing the production of a heterologous protein.

In one embodiment of the invention, the cell contains heterologous DNA encoding a mammalian serum protein. This particular embodiment when applied to plant, fungal or bacterial cells is particularly advantageous in that the risk of pathogen-contaminated blood products, such as contamination with hepatitis and/or HIV, is mitigated. It is envisaged that serum proteins produced by the method of the present invention will provide an alternative and safe source of donor serum and blood factors.

In other embodiments of the invention the cells contain heterologous DNA encoding, for example and without limitation, hormones such as human growth hormones and endocrine products such as insulin and thyroxine. As previously mentioned production of such hormones by the method of the present invention will provide a pathogen free product, especially when applied to production by to plant, fungal or yeast cells.

It is believed that proteolysis occurs in the lytic vacuole of plants or the central vacuole of micro-organisms such as fungi or yeasts. The present invention relates to a method to overcome or limit proteolysis by either preventing export of proteins out of the ER and/or by redirecting proteins from the vacuolar sorting route back to the ER or the cell surface, and in this way the yield of heterologous protein expression is increased.

We provide evidence that prevention of ER export leads to drastic increases in the total yield of protein, without the requirement of increasing the synthesis rate of the protein. We have shown increases in yield which are not marginal but surprisingly can be up to a hundred fold. This result is most surprising, because it involves the disruption of an essential biochemical pathway which would not be expected to be beneficial to an organism.

Preferably, limiting/preventing export of proteins out of the ER is by inhibition of COPII transport. This type of ER export is conserved in all eukaryotic cells and thus represents a universal target for inhibition.

Preferably, inhibition of COPII transport is achieved by either of the following methods:
(i) inhibition of COPII dependent vesicle budding via overproduction of the Sar1-specific guanosine exchange factor Sec 12;
(ii) co-expression of a mutant GTPase Sar1 which is less sensitive to its GTPase activating protein and as a result is defective in GTP hydrolysis.

Preferably, the exchange factor is Sec12p or an isoform thereof.

Preferably, the mutant GTPase is Sar1 or ARF1 or isoforms thereof.

Both proteins have equivalents in other eukaryotic cells. Plant Sec 12 and Sar1 homologs are so similar to their yeast counterparts that they can functionally complement the yeast homologs (d'Enfert et al., 1992).

Preferably, re-directing proteins from the vacuolar sorting route back to the ER may be achieved by co-expressing a modified vacuolar sorting receptor which carries an engineered ER retention signal and which is transported from the Golgi apparatus back to the ER instead of on towards the vacuolar compartment.

For example, in plants preferably the receptor is BP80 or a close isoform of said receptor and preferably the engineering of the ER retention signal is such that it does not interfere with its ligand binding properties.

In other eukaryotic cells, vacuolar sorting receptors may be modified in similar ways to achieve the same effect. The closest relative in yeast to BP80 is thought to be the VPS10 gene product and also mammalian cells contain receptor proteins which bind to ligands in the Golgi apparatus and which initiate targeting to the vacuole. It will thus be appreciated that any modified receptor molecule, modified in such a way as to re-direct proteins back to the ER instead of directing them to the lytic compartment are included within the scope of the present invention.

In one embodiment of the invention, the cell is adapted so that the BP80 phenotype is depleted by producing a BP80-mycHDEL receptor. We have found that BP80-mycHDEL is capable of retrieving proteins destined for the vacuole.

In an alternative embodiment of the invention BP80 is specifically engineered so as to reach the cell surface with its protein cargo, so that the protein may be secreted at the cell surface.

Preferably, the method of re-directing vacuolar proteins to the cell surface is via co-expression of a GTP-restricted form of the low molecular weight GTPase ARF1. For example, in plants, such over-expression causes transient secretion of those vacuolar proteins destined to the lytic vacuole via the BP80-route. If applied at the correct concentration and prior to harvesting of cells, such treatment will re-direct a significant proportion of the proteins normally destined to the lytic vacuole towards the cell surface and the extra-cellular fluid, in which many heterologous proteins would be stable. Purification from the culture medium would be easier, and combined with the higher stability and yield of the gene product this would constitute an immediate advantage.

Preferably, cells containing heterologous DNA are incubated in appropriate fermentation/incubation media for sufficient time to generate enough cell mass, subsequently the cells are subjected to either one or a combination of the previously described proteolysis inhibition treatments prior to harvesting of the heterologous protein.

Typically cells are subjected to either one or both of the proteolysis treatments for 12 to 48 hours prior to harvesting, but this may depend on the gene-product and should not be regarded as an exclusive recommendation or a limiting factor of the method of the invention.

It will be appreciated that other methods that control/regulate proteolysis are equally applicable and are intended to be included in the scope of the present application. For example, and without limitation, the disposal of malfolded proteins after retrograde transport from the ER to the cytosolic proteasome, which has been well described in mammals and yeast, may also be expected to operate in plants in addition to the vacuolar degradation. For instance, if prevention of ER export does not stabilise the heterologous protein in question (Example 1), retrograde translocation to the cytosolic proteasome could be prevented via mutant Sec61 overexpression. Similarly, vacuolar transport could also be re-directed to the cell surface instead of the endoplasmic reticulum (Examples 2 and 3).

Numerous ways of manipulating the secretory pathway could be envisaged to limit proteolysis. The invention is based on the surprising discovery that proteolysis and not synthesis is the limiting factor in heterologous protein production. Therefore, the three examples given should not be seen as an exclusive list of approaches.

Preferably, the method of the present invention will be utilised for large scale production of heterologous proteins. It is envisaged that plant/fungal/yeast/mammalian cells containing appropriate heterologous DNA encoding the protein of choice will be incubated in fermentation vats under suitable conditions and that the heterologous protein will be harvested therefrom. It is believed that the method of the present invention will provide improved yields in heterologous protein expression and, when used to produce serum/blood proteins mitigate problems associated with pathogen contaminated blood products.

Preferably, the method of the present invention further comprises any one or more of the following steps:
(i) cell culture;
(ii) harvesting;
(iii) purification;
(iv) modification;
(v) formulation; or
(vi) lyophilisation.

It will be appreciated that some of the heterologous protein produced by the present invention may be subjected to post synthesis modifications such as glycosylation or glycan modification.

Preferably, the formulation step includes providing the heterologous protein in a suitable diluent, carrier or exipient or alternatively the product may be freeze dried/lyophilised for subsequent use.

According to a further aspect of the invention there is provided a method of producing heterologous mammalian proteins comprising:
(i) incubating cells containing heterologous DNA encoding the protein of choice in appropriate incubation media until sufficient cell mass is generated;
(ii) reducing export of proteins out of the ER and/or re-directing proteins from the vacuolar sorting route back to the ER or the cell surface and;
(iii) harvesting the heterologous protein.

Preferably, the method further includes the step of disposal of remaining cell debris containing genetically modified material for example by fermentation to methane and/or incineration of solid material.

The method of the present invention is thus advantageous in a total containment strategy.

Preferably the method further includes any of the preferred features hereinbefore described.

According to a yet further aspect of the invention there is provided a method of producing heterologous mammalian serum proteins comprising:
(i) incubating cells containing heterologous DNA encoding the serum protein of choice in appropriate incubation media media until sufficient cell mass is generated;
(ii) reducing export of proteins out of the ER and/or re-directing proteins from the vacuolar sorting route back to the ER and;
(iii) harvesting the heterologous serum protein.

It will be appreciated that mammalian serum proteins is intended to include blood clotting factors, immunoglobulins and other such blood components.

Preferably, the method further includes any one or more of the features hereinbefore described.

According to a yet further aspect of the invention there is provided a protein product produced by the method of the present invention.

Preferably the method of producing the product further includes any of the preferred features hereinbefore described.

The invention will now be described by way of example only with reference to the following Figures wherein:

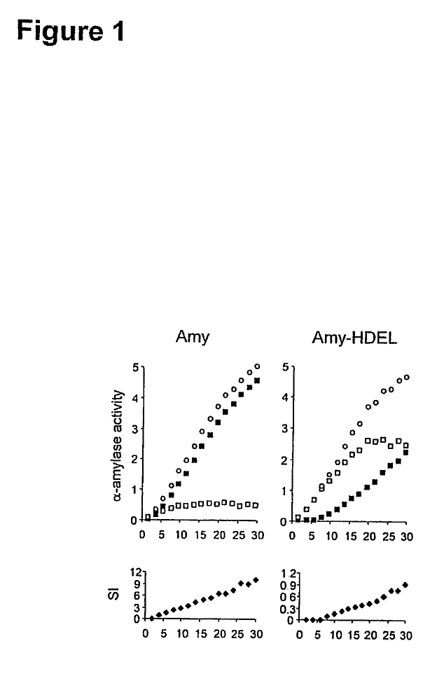

FIG. 1 illustrates Amy-HDEL secretion;

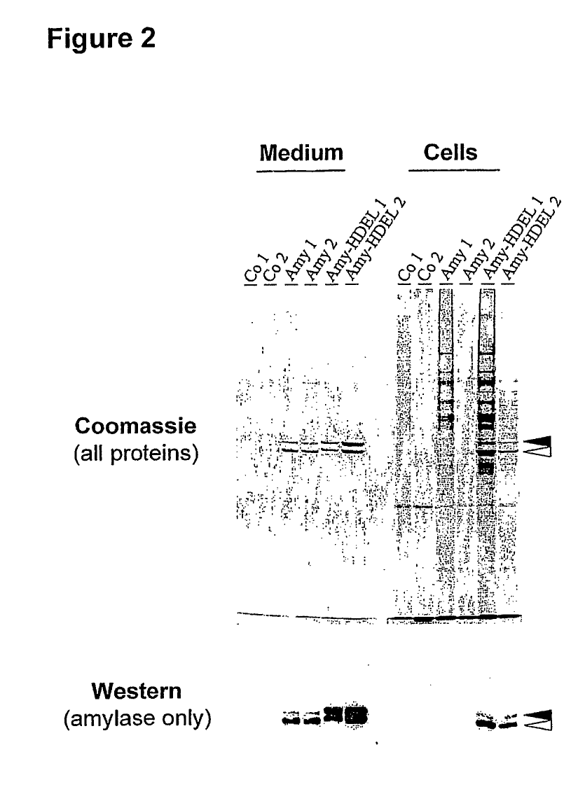

FIG. 2 illustrates Amy-HDEL secretion in stably transformed tobacco suspensions;

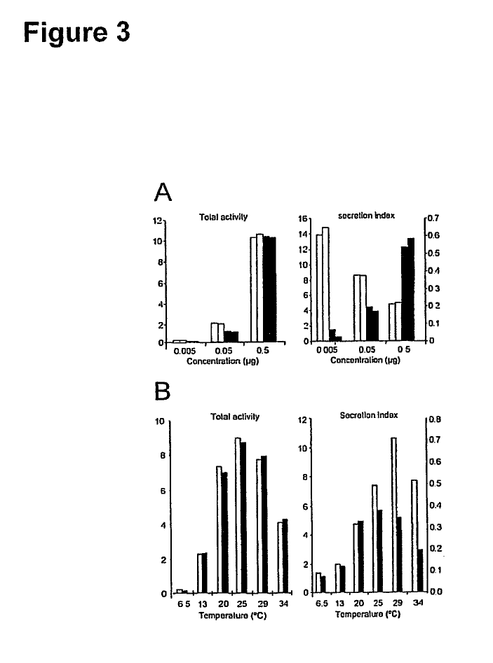

FIG. 3 illustrates the effect of cargo dosage and the temperature on secretion and retention;

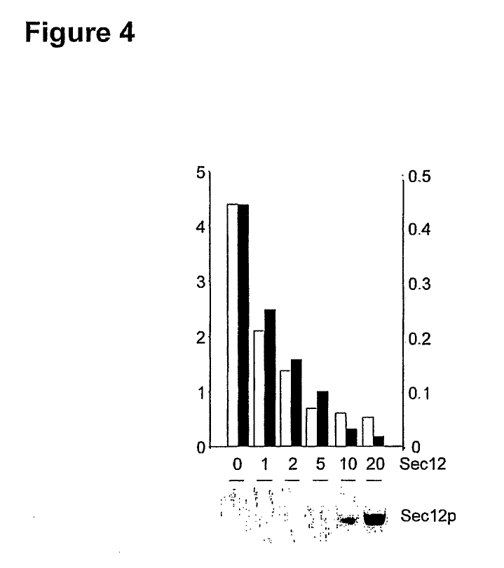

FIG. 4 illustrates in vivo manipulation of COPII dependent ER export;

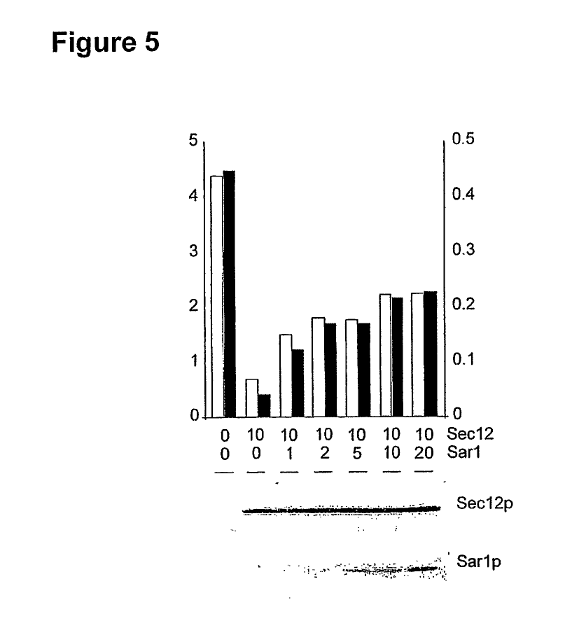

FIG. 5 illustrates evidence that Sar1 can restore Sec12 mediated inhibition of secretion;

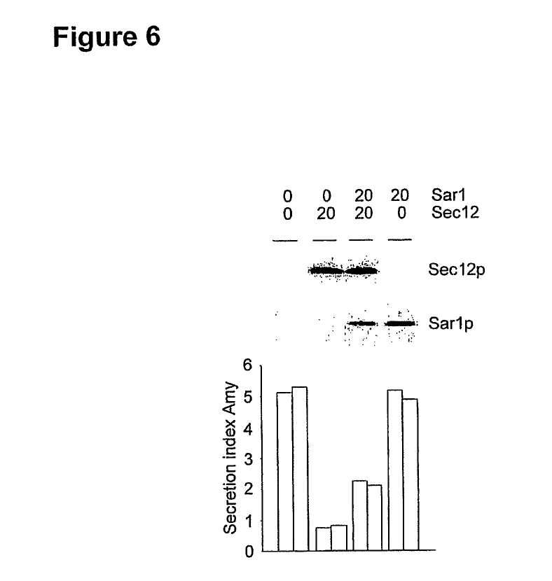

FIG. 6 illustrates evidence that Sar1p overexpression alone does not influence secretion;

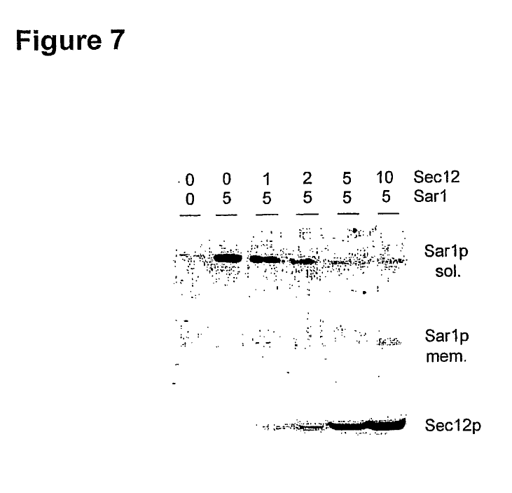

FIG. 7 illustrates membrane recruitment of Sar1p by increasing the levels of Sec12p;

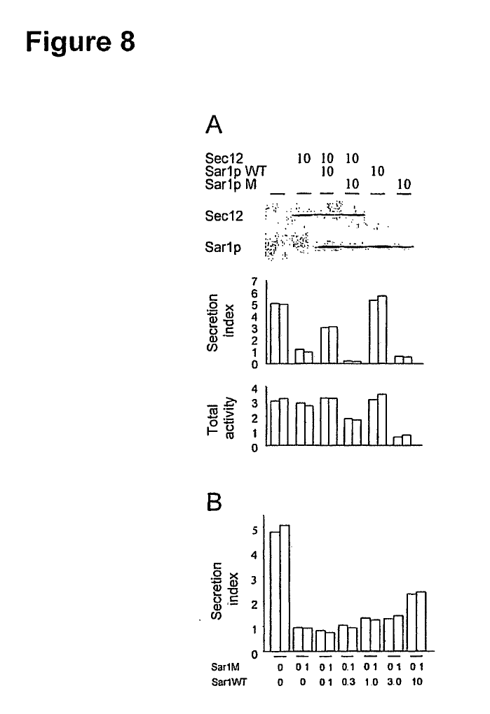

FIG. 8 illustrates inhibition of ER-Golgi transport using mutant Sar1 and comparison with Sec12;

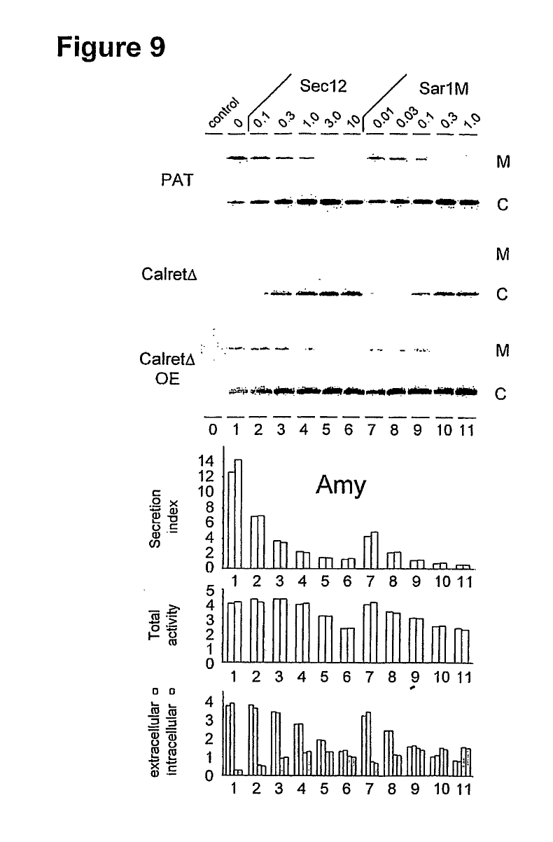

FIG. 9 illustrates secretion of ER residents and bull flow markers is COPII dependent;

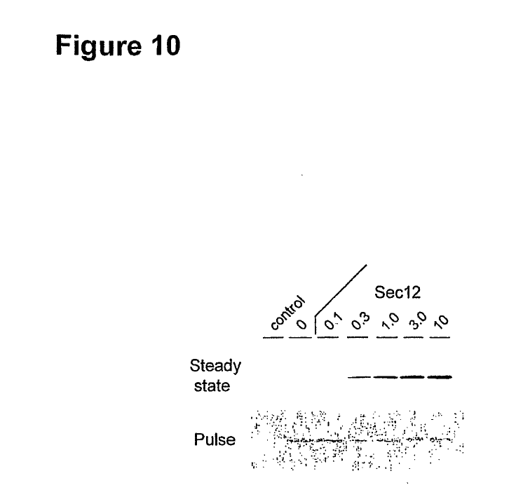

FIG. 10 shows a comparison of steady state protein levels and the de novo synthesis rate;

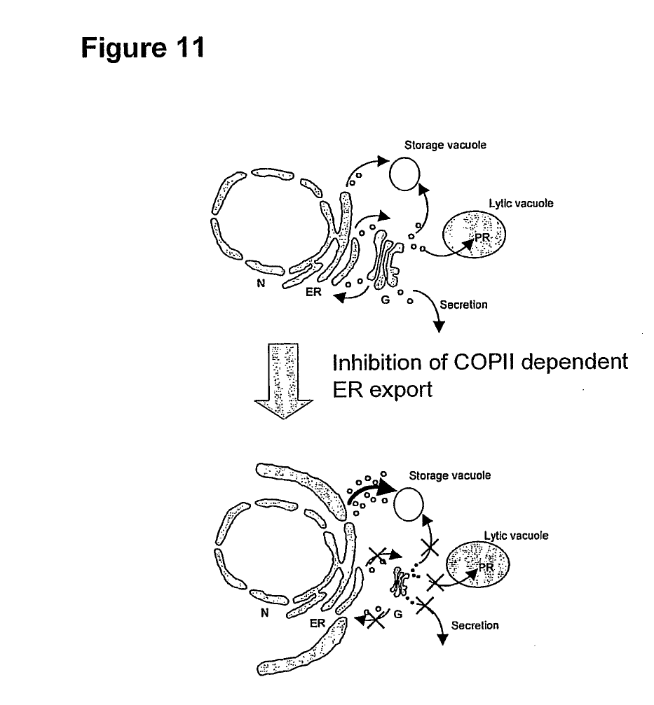

FIG. 11 illustrates Example 1;

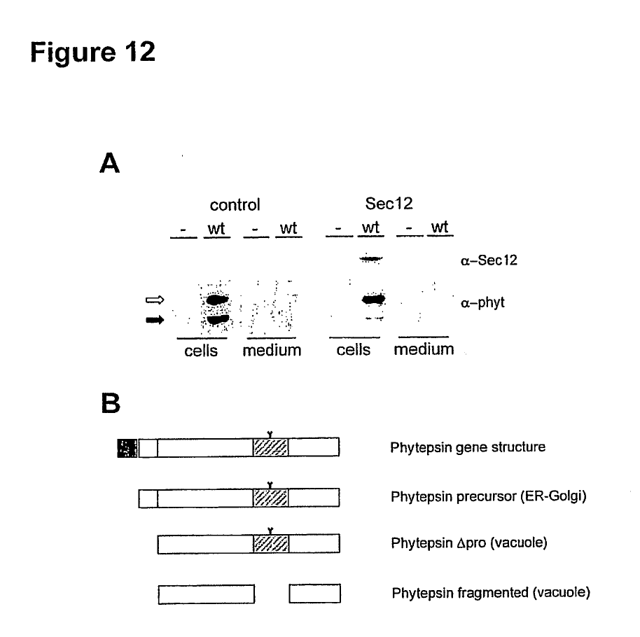

FIG. 12 illustrates transport of the vacuolar marker phytepsin;

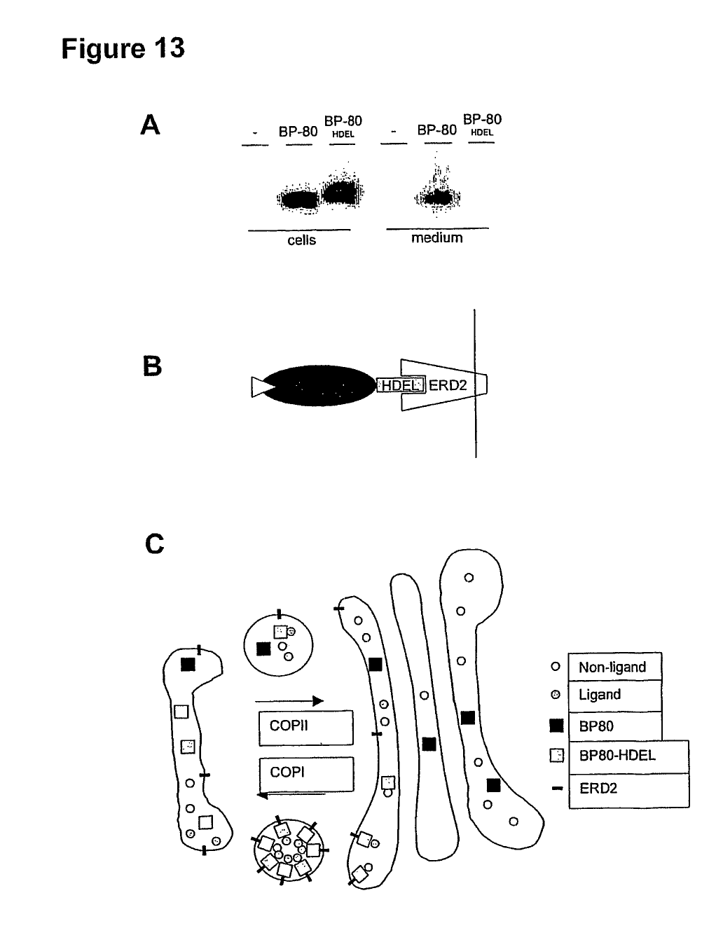

FIG. 13 illustrates transport of soluble BP80 and HDEL tagged soluble BP80;

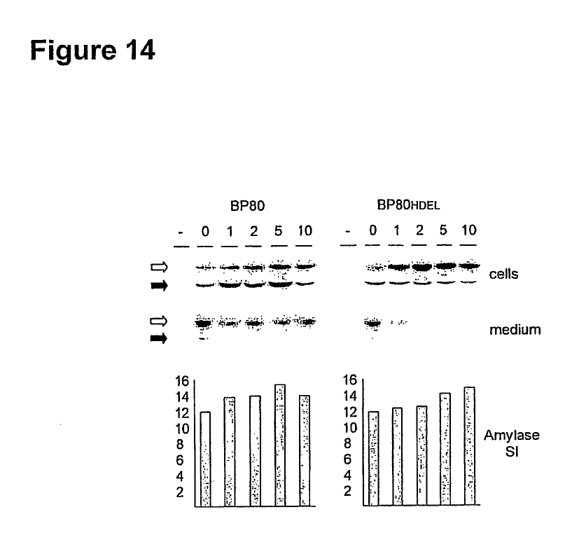

FIG. 14 shows that BP80-HDEL can cause specific accumulation of un-processed phytepsin in the ER without affecting a non-ligand for BP80 (α-amylase);

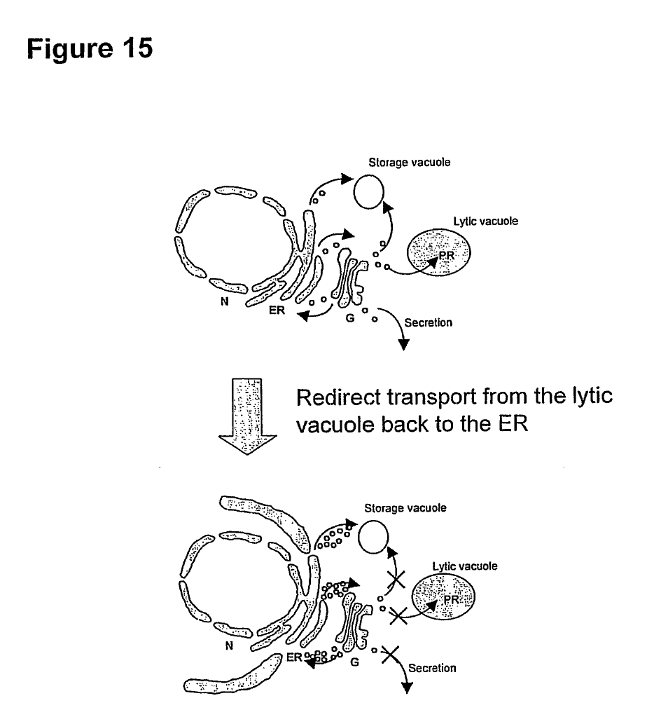

FIG. 15 illustrates Example 2;

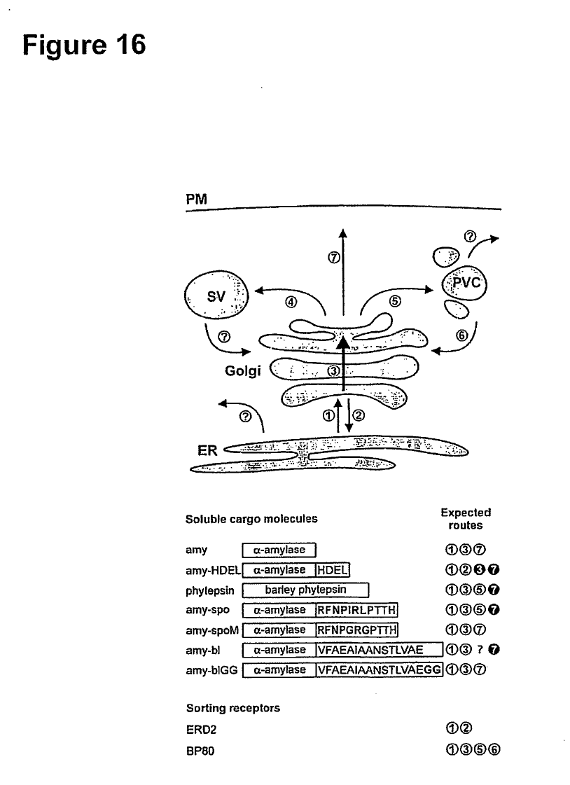

FIG. 16 illustrates a model predicted transport routes;

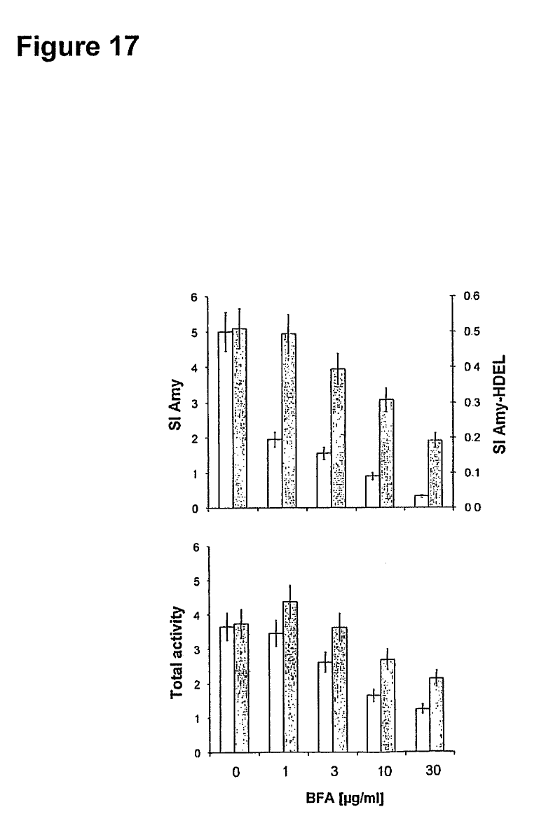

FIG. 17 shows the influence of increasing BFA concentration on the transport of amy and amy-HDEL in transient expression;

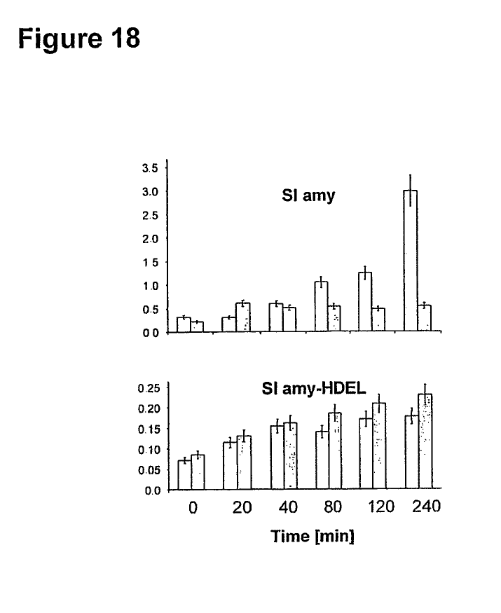

FIG. 18 shows the influence of BFA on the transport of amy and amy-HDEL in stable transformants in function of the time;

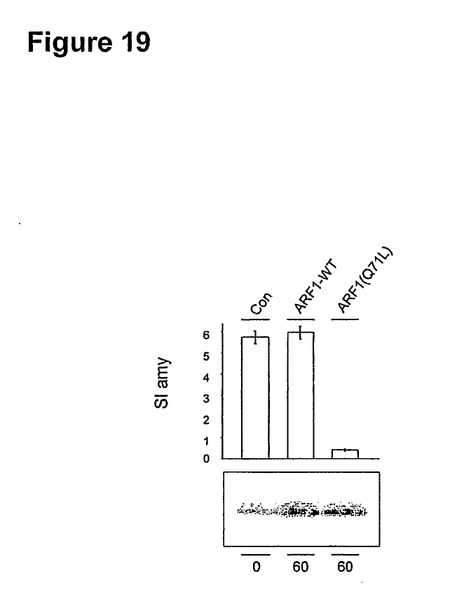

FIG. 19 shows transient expression of recombinant ARF1p and ARF1(Q71L)p in tobacco protoplasts;

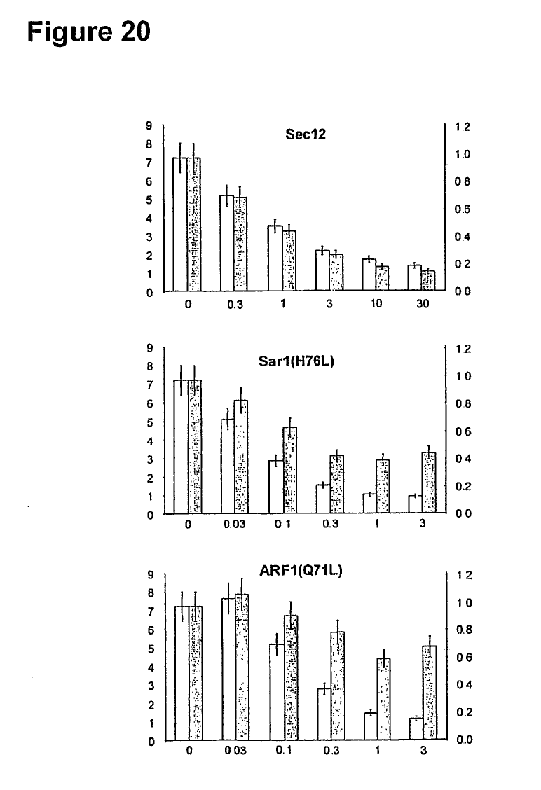

FIG. 20 shows the influence of Sec12, Sar1 and ARF1 on anterograde and retrograde transport;

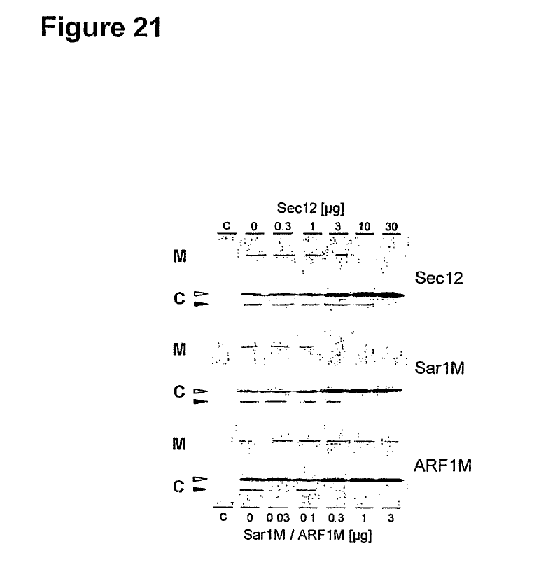

FIG. 21 shows the influence of Sec12, Sar1 and ARF1 on the transport of barley phytepsin;

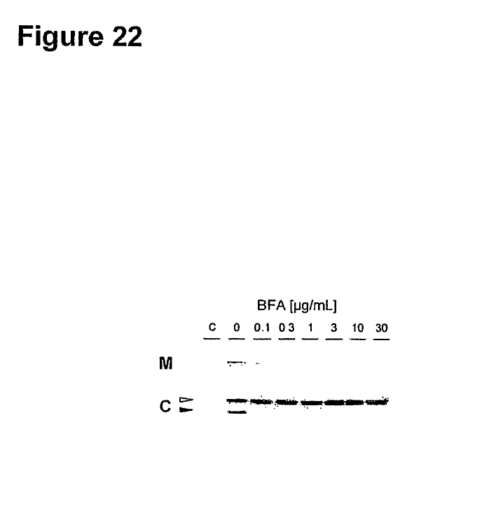

FIG. 22 shows the influence of increasing BFA concentration on the transport of phytepsin;

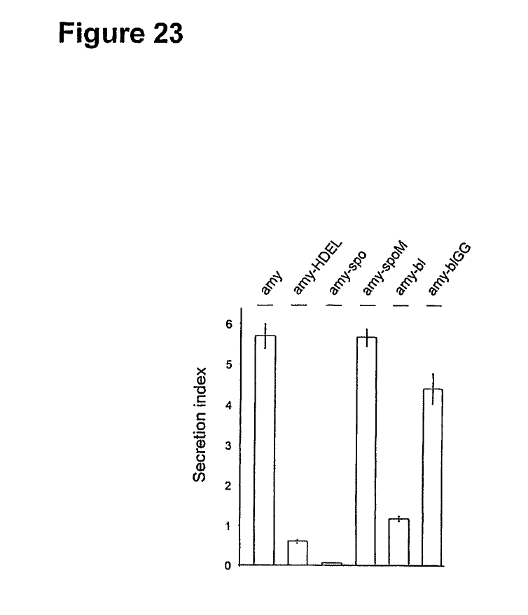

FIG. 23 illustrates the propeptides of sweet potato sporamin and barley lectin are functional at the C-terminus of α-amylase;

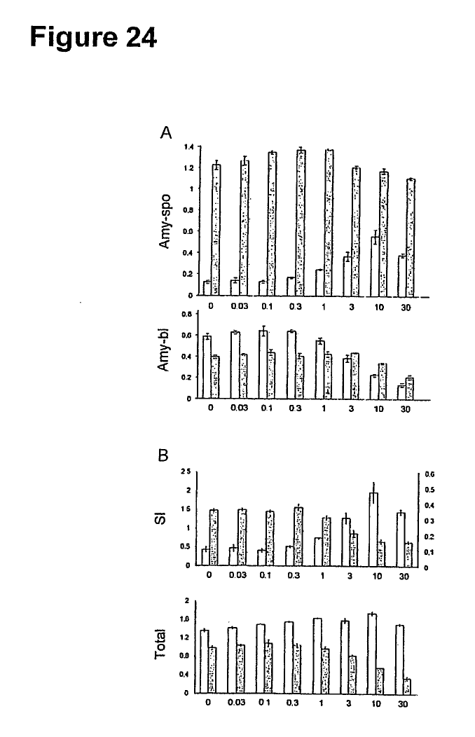

FIG. 24 shows ARF1(Q71L)-induced secretion specifically occurs for sequence specific sorting signals; and

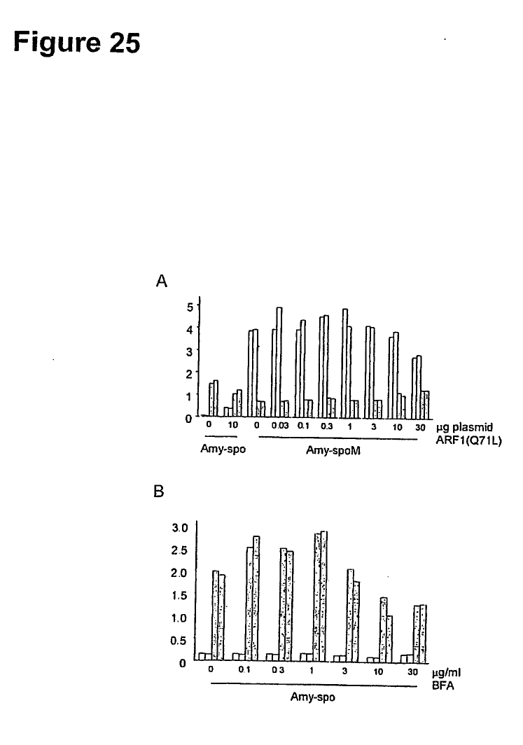

FIG. 25 shows ARF1(Q71L)-induced secretion can not be mimicked by BFA and is dependent on the NPIRL motif.

### DETAILED DESCRIPTION OF THE INVENTION

With regard to mole detailed description of the Figures:

FIG. 1. Amy-HDEL Secretion is Due to Saturation of the HDEL Receptor.

Time course of Amy (left) and Amy-HDEL (right) expression, showing α-amylase activity in cells (open squares), in the culture medium (black squares) and the total activity (circles). The lower panel shows the ratio between the extracellular and the intracellular α-amylase activity, termed secretion index (SI). Note the ten-fold difference in the scales.

FIG. 2. Amy-HDEL Secretion in Stably Transformed Tobacco Suspensions:

Medium and cells analysed via Coomassie stained protein gels or Western blot analysis. Control cells are untransformed, and amy producers are compared with amy-HDEL producers. All cultures were analysed 1 week after inoculum (1) or two weeks after inoculum (2). Note that both proteins are well recovered in the medium, but that the presence of HDEL mainly results in an increase in the cellular levels.

FIG. 3. Effect of Cargo Dosage and the Temperature on Secretion and Retention

(A) Total activity and Secretion index of Amy (white bars) compared to Amy-HDEL (black bars) measured 24 hours after transfection of protoplasts. The concentration of the plasmids used for transfection is indicated. The SI of Amy is given on the left y-axis while the SI of Amy-HDEL is given on the right y-axis.

(B) Influence of the temperature on protein synthesis (total activity) and the secretion index of Amy (white) compared to Amy-HDEL (black). The SI of Amy is given on the left y-axis while the SI of Amy-HDEL is given on the right y-axis.

FIG. 4. In Vivo Manipulation of COPII Dependent ER Export

Dosage dependent inhibition of secretion of Amy and Amy-HDEL by Sec12p co-expression measured 24 hours after transfection. The amount of Sec12p encoding plasmid is given in micrograms and a constant amount of Amy (white bars) or Amy-HDEL (black bars) encoding plasmids (2 μg) was used in each lane. The SI of Amy is given on the left y-axis while the SI of Amy-HDEL is given on the right y-axis. The western blot shows the expression of Sec12p in each lane.

FIG. 5. Evidence that Sar1 can Restore Sec12 Mediated Inhibition of Secretion

Partial reconstitution of Sec12p mediated inhibition of secretion by co-expression of Sar1p. Annotations are as in FIG. 4. The western blot illustrates the constant levels of Sec12p and the increased expression of Sar1p (concentrations of Sar1 encoding plasmid given in μg).

FIG. 6. Demonstration that Sar1p Overexpression Alone does not Influence Secretion.

Annotations are as in B), but only the secretion of Amy was tested with maximum levels of Sec12p and Sar1p.

FIG. 7. Membrane Recruitment of Sar1p by Increasing the Levels of Sec12p.

Protoplasts were extracted by osmotic shock to yield the cytoplasmic fraction (sol.) and the membrane fraction (mem.). Sec12p was only present in the membrane fraction. Quantities of plasmids are given in μg. Note that at high expression levels of Sec12, Sar1 is recruited to the membrane fraction at the expense of the cytosolic localisation.

FIG. 8. Inhibition of ER-Golgi Transport Using Mutant Sar1 and Comparison with Sec12

(A) Transport assay to detect the influence of the dominant negative GTP-trapped mutant of Sar1 after 24 hours of co-expression. The numbers above the lanes refer to the amount in μg of plasmids encoding either Sec12-, Sar1 wild type (Sar1p WT), or mutant Sar1 (Sar1p M). The upper panel shows a protein gel blot to detect the three different co-expressed proteins. The lower panel shows the secretion index and total activity respectively, corresponding to two independent transport assays using the secretory market α-amylase. Lanes are as in the upper panel. Note the strong reduction in the secretion index when both Sec12 and mutant Sar1 are co-expressed. Note also that Sar1p M alone causes a reduction in the total yield of α-amylase, and that further inhibition of secretion by superimposing increased Sec12 levels restores some of the yield. (B) Transport assay to monitor the effect of co-expressed wild type Sar1 on a constant level of mutant Sar1. Shown is the secretion index and the lanes indicate the quantities of the corresponding plasmids transfected in μg. Note that only a hundred fold excess of wild type Sar1 rescues some of the secretion inhibited by the mutant GTPase.

FIG. 9. Secretion of ER Residents and Bulk Flow Markers is COPII Dependent.

The culture medium (M) and the cells (C) of protoplast suspensions were recovered 48 hours after transfection and cargo molecules were detected either by protein gel blots (upper panels) or via enzymatic analysis (lower panels). The numbers above the lanes refer to the amount of Sec12p encoding plasmid (Sec12) or mutant Sar1 encoding plasmid (Sar1M) in μg. As cargo molecules, the bulk flow marker PAT or CalreticulinΔHDEL (CalretΔ) were co-expressed in constant amounts (2 μg). CalreticulinΔHDEL was also 10-fold over-expressed (CalretΔ OE) in the bottom panel (20 μg). The control lane is devoid of any cargo molecule. Note that calreticulinΔHDEL secretion is only seen when the protein is overexpressed. The lower panel shows the effect of the various inhibitory levels of Sec12 and mutant Sar1 on the transport of the secretory marker α-amylase. Shown is the secretion index, the total activity, and the activity in the cells (grey) and the culture medium (white) as indicated and lanes correspond to the upper panel. Note that the total α-amylase activity is slightly reduced with increasing levels of either transport inhibitors. Note also that the intracellular accumulation of α-amylase is not higher as that of PAT and calreticulinΔHDEL.

FIG. 10: Comparison of Steady State Protein Levels and the De Novo Synthesis Rate.

Tobacco protoplasts were harvested 24 hours after transfection and equal portions were analysed for protein levels (Steady state) using protein gel blots, or the ability to synthesize protein de novo during a 30 minute pulse labelling procedure (Pulse), detected via quantitative immunoprecipitation, subsequent SDS-PAGE and autoradiography. The numbers above the lanes refer to the amount of Sec12p encoding plasmid (Sec12) in μg. CalreticulinΔHDEL was co-expressed in low amounts (2 μg) as in FIG. 6 which does not allow detection of protein in the medium. Note that detected levels using pulse labelling were constant in sharp contrast to the steady state protein levels.

FIG. 11: Illustration of Example 1.

Upon inhibition of COPII dependent ER export, the Golgi apparatus is expected to desintegrate and transport to distal locations is inhibited, followed by accumulation of proteins in the ER. This results in dilated ER and presumably alternative transport to the storage vacuoles, which have been hypothesised to simply be the dilated ER itself.

FIG. 12: Illustration of Transport of the Vacuolar Marker Phytepsin

(A) Transport of phytepsin to the vacuoles results in processing, whereas secretion (due to saturation of the vacuolar transport route) results in the detection of un-processed protein in the culture medium. Inhibition of COPII transport by Sec12 overproduction increases the amount of unprocessed phytepsin in the cells and abolishes secretion of the unprocessed form, thus indicating that phytepsin exits the ER through the COPII route.

(B) Illustration of the processing steps associated with phytepsin synthesis and transport.

FIG. 13. Transport of Soluble BP80 and HDEL Tagged Soluble BP80.

(A) Transient expression showing that secretion of soluble BP80 is prevented by HDEL tagging. (B) Illustration of HDEL tagged BP80 and its interaction with the HDEL receptor ERD2. (C) Illustration of how BP80-HDEL can be employed to recycle vacuolar proteins back to the ER instead to the vacuoles via the dominant effect of the overproduced soluble BP80 derivative.

FIG. 14. Demonstration that BP80-HDEL can Cause Specific Accumulation of Un-Processed Phytepsin in the ER without Affecting a Non-Ligand for BP80 (α-Amylase).

Transient expression of phytepsin and an increasing amount of either soluble BP80 or BP80-HDEL. Note that Secretion is abolished by BP80-HDEL and that the un-processed form accumulates in the ER.

FIG. 15: Illustration of Example 2.

Upon overexpression of BP80-HDEL, proteins normally destined to the vacuoles will recycle from the Golgi back to the ER and accumulate there. This results in dilated ER and presumably alternative transport to the storage vacuoles, which have been hypothesised to simply be the dilated ER itself.

FIG. 16: Model Illustrating Predicted Transport Routes

Schematic drawing illustrating current knowledge on defined transport steps towards and from the Golgi apparatus in plants. COPII dependent ER export (1) is balanced by retrograde COPII mediated transport (2), clathrin mediated transport (5) to the prevacuolar compartment is matched by a retrograde route (6) which is believed to be dependent on the retromer complex. Anterograde intra-Golgi transport (3) and transport to the storage vacuole (SV) (4) and secretion (7) to the plasma membrane (PM) is mediated by as yet un-known transport mechanisms. Unknown transport pathways (?) denote export from the PVC, recycling from the SV, as well as unusually large ER export vessels (Hara-Nishimura et al., 1998; Toyooka et al., 2000) as well as COPII independent ER export of phytepsinΔPSI (Törmäkangas et al., 2001).

Cargo molecules used in this study and known sorting receptors of the plant secretory pathway are listed, including the predicted transport routes followed. White numbers in dark field show transport routes only followed during saturation of receptor mediated routes. Amy-spo and amy-spoM represent α-amylase fusion proteins containing WT or mutated (M) forms of the sweet potato sporamin (Koide et al., 1997). Amy-bl represents an α-amylase fusion protein with the propeptide of barley lectin (Bednarek and Raikhel, 1991). A non-functional derivative based on the established C-terminal double glycin addition (Dombrowski et al., 1993) is denoted as amy-blGG. Since barley lectin and sweet potato sporamin were shown to be transported to the same kind of vacuole (Schroeder et al., 1993) via different sorting mechanisms (Matsuoka et al., 1995), it can not simply be postulated that amy-bl follows route 4. Therefore, the expected Golgi derived transport route is left open and is indicated by the symbol (?).

FIG. 17: Influence of Increasing BFA Concentration on the Transport of Amy and Amy-HDEL in Transient Expression.

The upper panel shows the secretion index (SI) of amy (open bars) and amy-HDEL (grey bars) in function of increasing concentration of Brefeldin A (indicated at the bottom in μg/ml final concentration). The left hand y-axis denotes the SI of amy, and the right hand axis denotes the SI of amy-HDEL. Note the much stronger reduction of the SI for amy compared to amy-HDEL. The lower panel shows the total activity (medium+cells) from the same experiments in the upper panel, given in change in OD per ml of protoplast suspension per minute. Note the slightly increased total activity for amy-HDEL when BFA is present.

FIG. 18: Influence of BFA on the Transport of Amy and Amy-HDEL in Stable Transformants in Function of the Time.

The upper panel shows the SI of amy from washed protoplast suspensions during a 240 minute period after incubation in the presence (grey bars) and absence (open bars) of 30 μg/ml BFA. The lower panel shows the same for amy-HDEL. Note that BFA inhibits secretion of amy, as seen by a lower SI when compared to mock-treated cell suspension, whereas this difference is not seen for amy-HDEL.

FIG. 19: Transient Expression of Recombinant ARF1p and ARF1(Q71L)p in Tobacco protoplasts.

Co-expression of the secretory marker α-amylase (amy) with either wild type ARF1p or mutant ARF1(Q71L)p. The upper panel shows the SI for amy under control conditions (con) in comparison with the co-expressed ARF1 proteins. The lower panel shows the total amount of endogenous ARF1p (con) in comparison with the higher protein level when 60 μg of ARF1- or ARF1(Q71L) encoding plasmid was electroporated. Note the profound effect of ARF1(Q71L)p on amy secretion in contrast to the wild type molecule.

FIG. 20: Influence of Sec12, Sar1 and ARF1 on Anterograde and Retrograde Transport.

Co-expression of amy and amy-HDEL with increasing concentrations of Sec12-, Sar1(H76L)- or ARF1(Q71L)-encoding plasmids. The effector plasmid concentrations are given below each lane. SI values for amy are given in open bars, values for amy-HDEL are given in grey bars. Standard errors are indicated for all measurements. The left hand y-axis denotes the SI of amy, and the right hand axis denotes the SI of amy-HDEL. Note that only Sec12p over-expression causes efficient inhibition of secretion of amy-HDEL, whereas the two trans-dominant GTPase mutants seem to inhibit mainly amy-secretion and have little effect on amy-HDEL transport.

FIG. 21: Influence of Sec12, Sar1 and ARF1 on the Transport of Barley Phytepsin

Co-expression of the vacuolar protein phytepsin with increasing concentrations of Sec12-, Sar1(H76L)- or ARF1(Q71L)-encoding plasmids. The effector plasmid concentrations are given below each lane. Equal volumes of medium (M) and Cell (C) samples are compared by protein-gel blotting. The open arrow denotes the un-processed pro-phytepsin, whereas the closed arrow denotes the lower molecular weight vacuolar form of phytepsin devoid of the N-terminal pro-peptide (Törmäkangas et al., 2001). Note that ARF(Q71L) co-expression causes an induced secretion of pro-phytepsin to the medium, whereas Sec12p and Sar1(H76L)p inhibit both secretion and vacuolar sorting.

FIG. 22: Influence of Increasing BFA Concentration on the Transport of Phytepsin.

Transient expression experiment showing the influence of BFA (indicated above each lane in μg/ml final concentration) on secretion to the medium (M), intracellular retention (C) and the partitioning between processed (closed arrows) and un-processed (open arrows) forms of phyteposin. Note that BFA inhibits both secretion and intracellular processing of phytepsin.

FIG. 23: The Propeptides of Sweet Potato Sporamin and Barley Lectin are Functional at the C-Terminus of α-Amylase.

Transient expression experiment showing the SI for amy, amy-HDEL, amy-spo, amy-spoM, amy-bl, and amy-blGG (see FIG. 1) Note that that intracellular retention of amy-spo is almost complete with hardly any evidence for saturation of the BP80-mediated sorting route, whereas amy-bl retention is leaky and comparable to that of amy-HDEL.

FIG. 24: ARF1(Q71L)-Induced Secretion Specifically Occurs for Sequence Specific Sorting Signals.

Transient expression experiment testing the influence of an increasing concentration of ARF1(Q71L)p on the transport of amy-spo and amy-bl. A) Extracellular (open bars) and intracellular (grey bars) activities (given in change in OD per ml of protoplast suspension per minute) shown for amy-spo and amy-bl in function of increased dosage of ARF1(Q71L)p. The plasmid concentration is given below each lane in μg. Note an increase in the extracellular level of amy-spo at high concentrations of ARF1(Q71L)p, in contrast to amy-bl whose secretion diminishes under those conditions. B) Data from A) represented as SI or Total activity (change in OD per ml of protoplast suspension per minute) for direct comparison between amy-spo and amy-bl. Amy-spo is denoted in light grey (right hand y-axis for SI) and amy-bl is denoted in dark grey (left hand y-axis for SI). Note the opposite behaviour of the SI for the two fusion proteins. Note also that the total amy-bl activity drops with increasing dosage of ARF1(Q71L)p while that of amy-spo increases slightly.

FIG. 25: ARF1(Q71L)-Induced Secretion can not be Mimicked by BFA and is Dependent on the NPIRL Motif.

A) Transient expression experiment showing the influence of an increasing concentration of ARF1(Q71L)p on the intracellular (grey bars) and extracellular (open bars) levels of amy-spoM (exhibiting a mutated sorting motif). The plasmid concentration is given below each lane in μg. The first two lanes are a positive control demonstrating the ability of ARF1(Q71L)p to induce secretion of the wild-type sporamin fusion. Note that none of the concentrations of ARF1(Q71L)-encoding plasmid leads to induced secretion of the mutant sporamin fusion (compare with FIG. 9A, upper panel). B) Transient expression experiment showing the influence of increasing concentration of BFA (indicated at the bottom in μg/ml final concentration) on the intracellular (grey bars) and extracellular (open bars) levels of amy-spo. Note that BFA does not induce the secretion to the medium as seen for ARF1(Q71L)p co-expression (compare to FIG. 9A, upper panel).

Materials and Methods

Plasmid Construction for Transient and Stable Expression

All DNA manipulations were done according to established procedures. The Escherichia coli MC1061 strain (Casadaban and Cohen, 1980) was used for the amplification of all plasmids. Plasmids encoding for x-amylase and α-amylase-HDEL were described (Crofts et al., 1999) To allow detection of transiently expressed calreticulinΔHDEL (Crofts et al., 1999) among endogenous wild type calreticulin, pDE314C (Crofts et al., 1999) was engineered to incorporate a c-myc tag just prior to the stop-codon, resulting in the plasmid pCalmyc. The Sec12 and Sar1 overexpression plasmids were generated through PCR amplification of the corresponding cDNA clones (d'Enfert et al., 1992) to result in coding regions placed in between the Cauliflower Mosaic Virus 35S promoter and the 3′ untranslated end of the nopaline synthase gene as in pDE314C (Crofts et al., 1999). Site-directed mutagenesis of the Sar1 coding region was carried out to exchange the histidine codon in position 74 with a leucine codon, using the following two PCR primers: SARH74L-sense (5′TTGATTTGGGTGGTCTTCAGATTGCTCGTAG3′) SEQ ID NO:1 and SARH74L-anti (5′CTACGAGCAATCTGAAGACCACCCAAATCAA 3′) SEQ ID NO:2, yielding plasmid pLL18

Previously established plasmids were used encoding α-amylase and α-amylase-HDEL (Crofts et al., 1999), and phytepsin (Törmäkangas et al., 2001). The four plasmids encoding the α-amylase derivatives amy-spo (pSLH44), amy-spoM, amy-bl and amy-blGG were generated by inserting annealed oligonucleotide pairs between the BglII and XbaI sites of the α-amylase encoding plasmid which overlap with the last codon and the stop-codon. The following oligonucleotide pairs were used to generate the C-terminally fused peptide encoding regions: spo-sense (5′GATCAGATTCAATCCCATCCGCCTCCCCACCACACA CTAACT3′) (SEQ ID NO 3:, spo-anti (5′CTAGAGTTAGTGTGTGGTGGGGAGGCGGATGGGATT GAATCT3′) (SEQ ID NO:4); spoM-sense (5′GATCAGATTCAATCCCGGTCG CGGTCCCACCACACACTAA CT3′) (SEQ ID NO:5); spoM-anti (5′CTAGAG TTAGTGTGTGGTGGGACCGCGACCGGGATTGAATCT3′) (SEQ ID NO:6); bl-sense (5′GATCGTTTTTGCTGAAGCTATTGCTGCTAATTCTACTCTTGCTTGCT GGATAAT3′), (SEQ ID NO:7); bl-anti (5′CTAGATTATTCAGCAACAAGAGTA GAATTAGCAGCAATAGCTTCAGCAAAAAC3′) (SEQ ID NO:8); blGG-sense (5′GATCGTTTTTGCTGAAGCTATTGCTGCTAATTCTACTCTTGTTGCTGAA GGTGGATAAT3′), (SEQ ID NO:9); blGG-anti (5′CTAGATTATCCACCTTC AGCAACAAGAGTAGAATTAGCAGCAATAGCTTCAGCAAAAAC3′) (SEQ ID NO:10). Underlined areas represent point-mutations or inserted codons.

A new Sar1(H74L) overexpression plasmid (pPP11) was created by sub-cloning the Sar1 coding region of pLL18 (Phillipson et al., 2001) as a ClaI-XbaI fragment into pLL4, which contains the Cauliflower Mosaic Virus 35S promoter, a spacer DNA flanked by ClaI and XbaI sites overlapping with the translation intitiation and stop codon, followed by the 3′ untranslated end of the nopaline synthase gene. An Arf1p overexpressing plasmid pPP5) was created by PCR mediated amplification of the ARF coding region using the oligonucleotides ARF1-sense (5′GATCACCATGGGGTTGTCATTCGG3′) (SEQ ID NO:11) and ARF1-anti (5′GCTAACTCTAGATCTATGCCTTGCTTGCGAT3′) (SEQ ID NO:12) from first strand cDNA prepared from 5 day old seedlings of Arabidopsis thaliana prepared according to established procedures (Denecke et al., 1995). To generate ARF1(Q71L), the following two oligonucleotides were used for site-directed mutagenesis of pPP5: ARF1MS (5′GGGATGTTGGGGGTCTCGACAAGATCCG TCCA3′) (SEQ ID NO:13) and ARF1MA (5′TGGACGGATCTTGTCGAGACCC CCAACATCCC3′) (SEQ ID NO:14) resulting in the derived plasmid pLL20. Underlined regions represent point-mutations. All constructions were verified by sequencing analysis. All constructions were verified by sequencing.

Plant Material and Growth Culture Conditions

Plants (Nicotiana tabacum cv Petit Havana; (Maliga et al., 1973)) were grown in Murashige and Skoog medium (Murashige and Skoog, 1962) and 2% sucrose in a controlled room at 25° C. with a 16-hours day length at the light irradiance of 200 μE/m²sec.

Transport Assays

Tobacco leaf protoplasts were electroporated as described (Denecke and Vitale, 1995), and plasmid concentrations used are given in the figure legends. Harvesting of cells and culture medium as well as the enzymatic assays were done as described previously (Denecke and Vitale, 1995; Crofts et al., 1999; Leborgne-Castel et al., 1999). Equal volumes of cells and medium were loaded on protein gel blots or analysed via enzymatic assays to determine the fraction of the marker proteins inside and outside of the cells and to calculate the secretion index (Denecke et al., 1990). Membrane recruitment was assessed by an osmotic shock procedure described previously (Denecke et al., 1990; Denecke et al., 1992).

Protein Extraction and Enzymatic Assays

Protoplasts were extracted in α-amylase extraction buffer (Crofts et al., 1999) via sonication for 5 seconds. In all cases, extracts were cleared by 10 min centrifugation at 2500 g at 4° C. and the supernatant was recovered. α-amylase activity was determined as described (Crofts et al., 1999).

Protein Gel Blotting

Samples were loaded after twofold dilution with 2×SDS-PAGE loading buffer (200 mM Tris-Cl, pH 8.8, 5 mM EDTA, 1 M sucrose, and 0.1% bromophenol blue). Proteins in SDS-polyacrylamide gels were transferred onto a nitrocellulose membrane and then blocked with PBS, 0.5% Tween 20, and 5% milk powder for 1 hr. The filter was then incubated in blocking buffer with primary antibody at a dilution of 1/5000 for anti-PAT antibodies, anti-c-myc antibodies (Santa Cruz Biotechnology), whereas 1/2500 dilutions were used for anti-Sec12 (Bar-Peled and Raikhel, 1997) and anti-Sar1 antibodies (Pimpl et al., 2000). All antisera were from rabbit and incubation of secondary antibodies and further steps were done as described (Crofts et al., 1999).

Pulse Labelling and Immunoprecipitation

10⁶ protoplasts were labelled as described previously (Crofts et al., 1998), except that the concentration of labelled methionine was higher (200 μCi/mL). Immunoprecipitation of myc-tagged calreticulinΔHDEL was carried as described previously (Crofts et al., 1998), but using polyclonal anti-c-myc antibodies (Santa Cruz Biotechnology).

Immunocytochemistry

Preparation of ultrathin cryosections from tobacco root tips was carried out as described previously (Pimpl et al., 2000). Immunogold labelling was performed with Protein A Sepharose purified barley α-amylase antiserum diluted 1/100, kindly provided by Birte Svensson (Carlsberg Laboratory, Copenhagen, Denmark). Labelled sections were observed in a Philips CM10 electron microscope (Philips, Eindhoven) operating at 80 kV.

### EXAMPLE 1

Partial Secretion of α-Amylase-HDEL is Due to Saturation of the ER Retention Machinery

The addition of an ER retention motif can be deleterious to protein function as it implies a protein modification of the protein itself. Moreover, we demonstrate here that retention is incomplete and subject to saturation of the receptor. The transport of α-amylase was compared with that of the transport of a modified enzyme carrying the ER retention signals HDEL at its C-terminus (α-amylase-HDEL). At different time-points after transfection, the culture medium and cells were harvested and α-amylase activity measured.

FIG. 1A shows that α-amylase was rapidly secreted as judged by the low intracellular steady state level reached as a result of an equilibrium between de novo synthesis and export. Extracellular α-amylase activity was detected soon after transfection and rose linearly after 6 hours of expression. In contrast, α-amylase-HDEL accumulated to high levels within the cells and only reached an intracellular steady state level 20 hours after transfection. Secretion of x-amylase-HDEL into the medium became apparent after 10 hours of expression. At later time-points, the secreted levels of the HDEL tagged enzyme increased and the slope of the curve approached that of α-amylase. At the end of the time course, secretion of both enzymes was thus comparable.

The results demonstrated that the secretion of α-amylase-HDEL was due to saturation as a result of high intracellular levels. If partial secretion were due to incomplete presentation of the HDEL motif at the C-terminus of α-amylase, secretion would have occurred immediately following synthesis. However, FIG. 1 shows that retention was complete as long as the intracellular levels did not reach a certain threshold.

It should be noted that the total activity obtained was independent of the presence of the HDEL motif. This shows that the enzyme was stable and enzymatically active regardless of its position in the secretory pathway. In contrast, the ratio of the extracellular activity to the intracellular activity, termed the secretion index (Denecke et al., 1990), was dramatically decreased by tagging with the retention motif, as shown in FIG. 1B. The secretion index rose linearly over time for both cargo molecules but was generally an order of magnitude lower for the HDEL tagged protein as illustrated by the scale of the y-axis. Due to the linear nature of these curves, the secretion rate of different cargo molecules can be compared simply via measurement of the secretion index at a single time-point, conveniently after 24 hours of incubation.

FIG. 2 illustrates that saturation of HDEL-mediated recycling also takes places in stably transformed tobacco cell suspensions. α-amylase was secreted to the culture medium and hardly detectable in the cells. α-amylase-HDEL was detected in the cells, but still efficiently secreted to the culture medium. This illustrates that saturation of the retention mechanism has taken place.

In conclusion, HDEL tagging is not sufficient to guarantee high fidelity ER retention, and saturation can be easily observed if the cargo molecule is stable in the post ER compartments. In case of heterologous protein expression, saturation of the ER retention mechanism may have been difficult to observe due to instability, and could thus be a reason for sub-optimal yields.

Saturation of ER Export by α-Amylase Overproduction

To confirm that secretion of HDEL ligands is cargo dosage-dependent and not due to a change in the physiology of the cells during prolonged incubation, a concentration series of plasmids was used for transfections and cells were incubated for the constant period of 24 hours. FIG. 3A shows that the secretion index for α-amylase-HDEL (black bars) increased with higher plasmid concentration. Since the incubation time was identical in all cases, the data are consistent with the saturation model.

Surprisingly, the control experiment using different concentrations of the plasmid encoding α-amylase (white bars) revealed that high dosage of the cargo molecule resulted in a reduction of the secretion index, suggesting that saturation of anterograde transport had also occurred. The fact that secretion can be saturated by overexpression suggests there is a limiting factor that can be saturated. The data for α-amylase-HDEL (FIG. 3A) are thus the result of superimposing two counteracting effects, the saturation of anterograde transport and the saturation of the HDEL receptor. The latter effect appears to be much stronger, hence the net increase in the secretion index for α-amylase-HDEL.

Differential Effect of Temperature on Anterograde and Retrograde Transport

To further optimise our transport assay, we tested the influence of the temperature on the transport of these two cargo molecules. FIG. 3B reveals that the temperature had a strong effect on the total amount of enzyme activity obtained, but that this effect was identical for both cargo molecules. In contrast, the secretion index was influenced in a differential fashion which was dependent on the presence or the absence of the HDEL motif.

Whereas α-amylase would follow the anterograde transport route, the HDEL tagged version will engage in both anterograde and retrograde transport between the ER and the Golgi. The results suggest that the efficiency of retrograde transport increases faster with higher temperature compared to the efficiency of anterograde transport. The differential effect of the temperature on these two molecules illustrates that anterograde and retrograde transport are different cellular processes supported by different molecular machinery.

In addition, there was no sharp low temperature block of secretion, in contrast to findings from mammalian cells, because α-amylase secretion improved gradually with increasing temperature. The results indicate that 25° C. may be the optimum temperature for both synthesis and transport

Sec12p Dosage Dependent Inhibition of α-Amylase and α-Amylase-HDEL Export

To show that α-amylase and its HDEL tagged derivative exit the ER in COPII vesicles, we took advantage of the fact that overproduction of Sec12p reduces ER export (d'Enfert et al., 1991b; d'Enfert et al., 1991a; Hardwick et al., 1992; Barlowe et al., 1993; Barlowe and Schekman, 1993; Nishikawa et al., 1994), presumably via the titration of Sar1 which is essential for COPII vesicle budding (Barlowe et al., 1993; Barlowe and Schekman, 1993; Barlowe et al., 1994). We used the Arabidopsis thaliana homologue of Sec12p which was shown to complement the corresponding yeast mutant (d'Enfert et al., 1992).

FIG. 4 shows that Sec12p overproduction resulted in a clear dosage dependent inhibition of α-amylase secretion. In the case of α-amylase-HDEL, inhibition of ER export via Sec12p overproduction would be expected to alleviate the saturation of the HDEL receptor as smaller amounts of the ligand reach the cis-Golgi. Indeed, FIG. 4 shows that at high levels of Sec12 a steeper inhibition of secretion for α-amylase-HDEL was observed, providing additional support for the saturation model.

Sec12p Overexpression Inhibits COPII Transport via Titration of the GTPase Sar1p

It has been suggested that the ER export inhibition via Sec 12p overexpression is due to titration of the low molecular weight GTPase, Sar1p (d'Enfert et al., 1991b; d'Enfert et al., 1991a; Hardwick et al., 1992; Barlowe et al., 1993; Barlowe and Schekman, 1993; Nishikawa et al., 1994), , thus leading to an inhibition of COPII vesicle budding which is dependent on Sar1 (Barlowe et al., 1993; Barlowe and Schekman, 1993; Barlowe et al., 1994). To provide experimental evidence for this claim, we attempted to rescue secretion of α-amylase via co-expression of increasing levels of Sar1p superimposed onto a constant inhibitory level of Sec12p. FIG. 5 shows that a partial reconstitution of α-amylase secretion did occur upon co-expression of Sar1p.

To rule out that this recovery was due to an independent effect of Sar1p overexpression on secretion, increasing levels of Sar1p were tested in the absence of Sec12p overexpression. In this case, no effect was observed on α-amylase secretion (FIG. 6). This result is very important as it shows that the recovery observed in FIG. 5 must have been due to a suppression of the Sec12p effect, and is not due to an independent effect of increasing Sar1 levels. This provides final proof of the titration hypothesis, for which no direct proof existed to date (d'Enfert et al., 1991b; d'Enfert et al., 1991a; Hardwick et al., 1992; Barlowe et al., 1993; Barlowe and Schekman, 1993; Nishikawa et al., 1994).

To demonstrate that Sec12p can physically interact with Sar1p or alter its cellular location, we co-expressed an increasing amount of Sec12p with a constant amount of Sar1p. FIG. 7 shows that Sar1p is mostly cytosolic when overproduced, but that increasing levels of Sec 12p result in recruitment of the GTPase to the membrane. The four experiments shown clearly establish how Sec12 overproduction can be used in plant cells as an elegant way to inhibit COPII transport.

Inhibition of COPII Transport via Co-Expression of Mutant GTP Trapped Sar1

To establish an alternative method to manipulate COPII transport in our system, we took advantage of the known trans-dominant negative effect on COPII vesicle transport by a GTP trapped mutant of Sar1p in vitro and in vivo (Saito et al., 1998; Takeuchi et al., 1998; Takeuchi et al., 2000). This mutant is less sensitive to the GTPase activating activity of Sec23, and results in stabilising vesicles in a coated configuration that is unable to fuse with the target membrane. FIG. 8A shows that in contrast to wild type Sar1p, mutant Sar1p inhibits α-amylase secretion when co-expressed. In addition, co-expression of Sec12 with mutant Sar1 causes a further reduction in secretion as compared to co-expression of either molecules alone, in contrast to wild type Sar1, which alleviates the effect of Sec12 overexpression. Both wild type and mutant Sar1 were detected in comparable levels using protein gel blotting, thus ruling out any artefact due to differences in the expression levels.

Interestingly, the Sar1 mutant caused a reduction in the total yield of the secretory marker. This cannot be due to a general toxifying effect of the GTPase mutant, because co-expression of Sec12 with the same amount of the mutant GTPase restored higher levels of the secretory marker. This occurred in spite of a further reduction in secretion. Sec12 overproduction alone also inhibits secretion without a significant change of the total yield. This means that the negative effect of the GTPase mutant on the total α-amylase levels was not due to the inhibition of the secretion process itself.

To further compare the two methods, we tried to establish whether the GTPase mutant inhibits COPII transport through a displacement of the wild type molecule. FIG. 8B shows that even one hundred fold lower levels of the mutant molecule as compared to those used in FIG. 8A could cause efficient inhibition of secretion, albeit with less loss of enzyme yield (data not shown). In contrast, co expression of the wild type protein had no significant rescuing effect at stoichiometric levels. Even when the level of wild type protein was increased, a rescuing effect was hardly noticeable, unless one hundred fold overexpression relative to the mutant was implemented. The results clearly illustrate the strong dominant negative effect of this mutant.

Together, the results suggest that the GTPase mutant inhibits the COPII transport mechanism at a different level compared to Sec 12, which acts through a depletion of Sar1. It is likely that Sec 12 overexpression inhibits the recruitment of the COPII coat which is dependent on Sar1, and thus acts at the earliest possible position in the pathway. In contrast, the GTPase mutant prevents uncoating of the vesicles and thus acts at a later stage. Both methods are thus independent approaches to manipulate COPII transport in vivo.

ER Export of a Bulk Flow Marker is COPII Dependent and Results in Degradation

In vitro generated COPII vesicles from yeast have been shown to contain anterograde cargo molecules such as yeast α-factor, but not ER residents such as BiP (Barlowe et al., 1994). However, bulk flow to the cell surface has repeatedly been shown to occur for a number of soluble passenger molecules in plant cells (Vitale and Denecke, 1999), albeit at low rates. This either suggests differences in the early secretory pathway between yeast and plants, or that in addition to COPII vesicles, other transport mechanisms exist to carry bulk flow out of the ER. To distinguish between these possibilities, we tested whether the secretion of bulk flow markers (Denecke et al., 1990) or ER residents (Crofts et al., 1999) is COPII dependent,

FIG. 9 shows that secretion of the bulk flow marker phosphinothricin acetyl transferase (PAT) (Denecke et al., 1990; Denecke et al., 1992) is inhibited by Sec12p overexpression as well as mutant Sar1 co-expression, demonstrating that PAT was transported in a COPII dependent fashion. In contrast to α-amylase, the increase in intracellular PAT levels exceeded the reduction in extracellular PAT. This was unexpected and suggests that a significant portion of PAT is degraded when allowed to proceed further than the ER in the secretory pathway.

ER Export of ER Residents is COPII Dependent and Results in Degradation

To test if ER residents are also transported in a COPII dependent fashion, a truncated form of calreticulin lacking its HDEL signal (calreticulinΔHDEL) (Crofts et al., 1999) was co-expressed with an increasing amount of Sec12p. FIG. 9 shows that secretion of calreticulinΔHDEL is only detected when the molecule is overexpressed, confirming earlier observations (Crofts et al., 1999). However, inhibition of COPII dependent ER export led to a dramatic recovery of intracellular calreticulinΔHDEL. This is particularly clear at low expression levels where no secretion is observed but Sec12 mediated recovery of calreticulinΔHDEL is very high. The latter result was even more surprising and suggests that most if not all the calreticulin molecules that escape from the HDEL mediate recycling are in fact degraded instead of transported to the cell surface.

In contrast to FIGS. 4-7, the experiment shown in FIG. 9 was done using a 48 hour incubation to maximise the effect of the inhibitors. To compare the data with those of FIGS. 4-7, we have also conducted the same experiment with the secretory marker α-amylase (FIG. 7, lower panel). Under these conditions, all secretion indices are approximately twice as high as in FIG. 4, as predicted from the linear relationship of the secretion index seen in FIG. 1. The longer incubation period allowed more time for Sec12 overproduction or mutant Sar1 production. This increases the sensitivity of the assay, which can be deduced from the smaller quantities of Sec12 or mutant Sar1 encoding plasmids used. In addition, the GTP trapped mutant can also exhibit a dosage dependent effect and reduces the total yield to a lesser extent when lower plasmid concentrations are used as compared to those in FIG. 8A. These results illustrate the quantitative nature of our transport assays and that co-expression of the secretory marker can be used as a routine method in conjunction with Sec12 or the Sar1 mutant to document inhibition of ER export.

Bulk Flow is Efficient and can Lead to Proteolysis

The data in FIG. 9 allow a direct comparison of the intracellular accumulation of the two test molecules PAT and calreticulinΔHDEL with that of the secretory marker α-amylase. It is shown that COPII transport inhibition leads to a similar, if not higher accumulation of the two test molecules. This is particularly clear in the case of calreticulinΔHDEL when it is expressed at low levels. Since neither inhibition strategy has led to an increase in the total yield of the secretory marker, it can be concluded that the capacity of the ER to synthesize proteins was not higher. Hence, the increase in the total levels of PAT and particularly calreticulinΔHDEL cannot be due to a higher synthesis rate or an indirect effect on the 35S promoter, which controls the transcription of these three cargo molecules.

One farther control experiment was conducted to rule out an increased synthesis rate of calreticulinΔHDEL as a result of the inhibition of ER export. At the intermediate time-point of 24 hours, when transient expression is still active but Sec12 overexpression already has a strong effect on the ER export process, we compared the steady state protein levels with the protein synthesis rate at that time-point. The former will have accumulated over the complete duration of the 24 hour period and depends on the rate of synthesis as well as the rate of degradation.

To estimate the synthesis rate, we conducted a pulse-labelling of the cells for 30 minutes starting at the 24 hour time-point. Subsequent quantitative immunoprecipitation and SDS-PAGE revealed the amount of protein synthesized during this short time-interval and provided an indication of the synthesis rate. FIG. 10 clearly shows that although the steady state protein levels are very much increased by the presence of Sec12, the amount synthesized during the 30 minute interval (pulse) was almost identical in all cases. This shows once more that Sec12 overexpression had no effect on the synthesis of calreticulinΔHDEL, and that the increase in the steady state level must be the result of preventing turn-over.

Comparison of FIGS. 9 & 10 reveals that at 24 hours, the lowest level of Sec12 has no rescuing effect, whereas at 48 hours a weak stabilisation is observed compared to the control. This again shows that longer incubations provide a more sensitive ER export inhibition assay, as could be predicted from the fact that it requires time to build up Sec12p to sufficient levels to titrate Sar1p.

We concluded that PAT and calreticulinΔHDEL are efficiently exported from the ER via COPII mediated transport, followed by degradation in a post-ER compartment. This prevents the molecule from reaching the culture medium and explains previous results on the poor secretion of ER residents when devoid of their ER retention signal or during saturation of the HDEL pathway. In addition to an explanation, it also offers a strategy to prevent degradation of heterologous proteins expressed in the secretory pathway of plants, which can lead to significant advances in biotechnology. FIG. 10 demonstrates that yields can increase from undetectable to significant levels, which is very attractive indeed from a commercial perspective. FIG. 11 illustrates the principle of example 1, which could indeed be of tremendous commercial value.

### EXAMPLE 2

Functional Demonstration of BP80 Functions in Planta

The transport pathway of wild type phytepsin is illustrated in FIG. 12 and shows that phytepsin follows the normal COPII dependent transport route out of the ER. We wanted to determine if phytepsin could bind to the putative vacuolar receptor BP80 (Kirsch et al., 1994). The binding properties of phytepsin have only been shown using in vitro binding studies using either affinity columns or cross-linking studies. Therefore, we wanted to determine if phytepsin can interact with BP80 in vivo, which will begin to demonstrate the biological relevance of this putative receptor. In order to achieve these goals we took advantage of the documented observations that the binding of BP80 can still occur at pH 7.0 even though it is slightly reduced compared to its optimal binding of pH 6.5 (Kirsch et al., 1994). Secondly, although the removal of the transmembrane domain and cytosolic tail of BP80 results in a secreted protein, these domains are dispensable for ligand binding (Cao et al., 2000). We took advantage of this feature and generated a soluble derivative of BP80 either with or without the ER retention motif HDEL. We also incorporated a c-myc tag within the two molecules so that our introduced protein could be easily distinguished from any endogenous BP80, which may cross react with the available BP80 antibodies. The Arabidopsis thaliana BP80 isoform with the highest homology to the pea BP80 was chosen for this purpose (Paris et al., 1997). FIG. 13A shows that BP80-myc is secreted and that HDEL tagging prevents this completely, showing that the c-myc tag is not interfering with the folding of the modified soluble BP80 molecules and that the HDEL signal is well presented on the surface.

To test if an interaction between phytepsin and BP80 occurs in vivo, we used the following strategy as described in FIGS. 13B-C to retrieve phytepsin from the normal route and deviate it towards the retrograde route back to the ER. We postulated that BP80 binding to its ligand may not only occur in the TGN, but also in all other Golgi compartments and perhaps even the ER based on findings on the pH dependence of BP80 ligand binding (Kirsch et al., 1996). In addition, we created a BP80 depleted phenotype by over expressing phytepsin and causing saturation of the pathway. Therefore, if a constant amount of phytepsin at saturating levels were co-expressed with increasing quantities BP80-mycHDEL, any interaction between the molecules would be observed as a reduction in secretion as well as a reduction in processing due to the continued recycling of the molecules due to the presence of the HDEL on BP80.

FIG. 14 illustrates that the HDEL tagged BP80 derivative is capable of retrieving an un-processed form of phytepsin from the exocytic pathway as concluded by the inhibition of secretion, reduced levels of processed (vacuolar) forms and increased levels of un-processed forms in the cellular fraction. No significant effect was observed for BP80-myc lacking the ER retention motif, suggesting that the retrieval was HDEL dependent and not due to non-specific aggregation. More importantly, co-expressing of a construct encoding the secretory marker α-amylase revealed that the interaction between phytepsin and BP80 is specific as neither BP80-myc nor BP80-mycHDEL influenced the secretion of α-amylase.

Therefore, we conclude that BP80-mycHDEL is capable of retrieving proteins destined to the vacuole and could thus form a suitable approach to prevent vacuolar mis-sorting of heterologous proteins as well. Likewise, BP80 could be specifically engineered to reach the cell surface and carry proteins, normally destined to the lytic vacuole, to the cell surface for secretion. The re-direction of a sorting receptor to an unusual location is used here as an example to illustrate how the secretory pathway can be engineered so as to limit proteolysis and lead the desired protein to the most suitable location for accumulation and subsequent recovery. This again has tremendous potential for commercial applications.

### EXAMPLE 3

Brefeldin A Inhibits Both Anterograde and Retrograde Transport

It is established that the molecular target of BFA is the guanosine nucleotide exchange factor for ARF1p, resulting in a stabilisation of an abortive complex with the GDP-bound form of ARF1p at the Golgi membrane. To test if inhibition of retrograde and anterograde transport can be dissected, we decided to utilise the secreted cargo molecule α-amylase (amy) and its derivative carrying an ER retention signal (amy-HDEL), which permits a quantitative analysis of transport rates in tobacco leaf protoplasts (FIGS. 1-6). We have shown before that the secretion index (SI), the ratio between extra- and intracellular activities, provides a simple way to compare relative transport rates at a defined time-point. In addition, amy and amy-HDEL have identical specific enzyme activity and stability throughout the secretory pathway. While both molecules are expected to have identical ER export properties (FIG. 16, route 1), only amy-HDEL can undergo retrograde transport from the Golgi (FIG. 16, route 2). If saturation of the HDEL receptor occurs, amy-HDEL is expected to reach the cell surface in the same manner as amy (FIG. 16, routes 3 and 7). We now tested the influence of different concentrations of BFA on the transport of either cargo molecules via transient gene expression in tobacco protoplasts. FIG. 17 shows that BFA causes a concentration-dependent inhibition of α-amylase secretion, visible even at very low concentration of the drug. In contrast, the slow secretion of the HDEL tagged derivative is hardly influenced by the drug, and only visible at high BFA concentrations. Corresponding well with previous findings (Crofts et al., 1999), BFA treatment causes a reduction in the total amount of α-amylase that is produced during the incubation period. This effect is less obvious for amy-HDEL.

If it is postulated that inhibition of ER export is equal for both cargo molecules, the results in FIG. 17 would indicate that BFA inhibits both anterograde and retrograde transport in the secretory pathway. Since amy-HDEL follows both transport routes, it would seem logical that it reacts relatively neutrally to the drug, less is transported to the Golgi (route 1), but less is recycled (route 2), resulting in roughly a similar release from the cells (routes 3 and 7). In contrast, amy will only follow the anterograde route, and the effect of inhibiting this route is easily detected even at low concentration of the drug.

The experiment shown in FIG. 17 was conducted using a standard transient expression assay with a 24-hour incubation period. This is a long time-period and it is possible that prolonged inhibition of the recycling of essential COPII vesicle budding components leads eventually to an inhibition of anterograde transport. To test if BFA affects anterograde transport only indirectly, it should be possible to show that BFA first causes a transient secretion of amy-HDEL before causing a collapse of the COPII anterograde route. To test this, we have carried out a transport experiment in which stably transformed tobacco cells were incubated for different time-periods in the presence of BFA. Under these circumstances, protoplasts contain a steady state level of amy or amy-HDEL from the very beginning of the experiment. This steady state level is very high for amy-HDEL, thus secretion of the cargo into the medium should be detected with high sensitivity if BFA first inhibits COPII retrograde transport while COPII anterograde transport is still efficient. FIG. 18 shows that BFA only inhibits the secretion of amy. However, the slow secretion of amy-HDEL is slightly induced after prolonged incubation (80 minutes and more) at which inhibition of amy secretion becomes significant. It was not possible to detect a transient induction of amy-HDEL secretion prior to the inhibition of amy secretion.

Sec12p Overproduction Specifically Inhibits Anterograde Transport from the ER

The results obtained in FIGS. 17 and 18 prompted us to revisit our previous findings on the inhibition of COPII transport using overexpression of the Sec12 gene product and co-expression of a trans-dominant negative mutant of the GTPase Sar1p carrying a point-mutation (H74L) that interferes with GTP hydrolysis (FIGS. 4-10). Although both approaches inhibited α-amylase secretion to a similar extent, the mechanism of inhibition occurs at different steps of the transport route. While Sec12p overproduction causes titration of the cytosolic Sar1p and thus inhibits COPII vesicle budding, the Sar1p GTPase mutant would interfere with vesicle uncoating and subsequent fusion with the Golgi. Interference with GTP hydrolysis has also been suggested to affect cargo loading (Nickel et al., 1998; Pepperkok et al., 2000), but this has not been shown for soluble proteins yet. We thus wanted to compare the effect of these inhibitors on both amy and amy-HDEL. To extend the available tools, we also generated an equivalent trans-dominant negative mutant of ARF1p carrying a point mutation (Q71L), which is GTP restricted (Pepperkok et al., 2000) and thus belongs to a similar class of mutation as the H74L mutant of Sar1p.

We have shown previously that Arabidopsis equivalents of Sec12p and Sar1p (d'Enfert et al., 1992) can be detected in electroporated tobacco protoplasts and distinguished from the endogenous tobacco gene products when expression is high. FIG. 19 shows that over expression of Arabidopsis ARF1p is difficult to detect above endogenous levels, although possible when using high plasmid concentrations. This is possibly due to the higher degree of conservation of ARF1 compared to Sar1 among eukaryotes (Pimpl et al., 2000). Given the fact that only a small percentage of protoplasts is transfected with plasmids, the difference seen in FIG. 19 shows that over expression must be significant in the transfected cells. Interestingly, over expression of wild type ARF1p does not influence amy-secretion, whereas the GTP restricted mutant has a very strong inhibitory effect, similar to previous observations with Sar1p before (Phillipson et al., 2001). This means that ARF1p does not only influence retrograde transport, which should not be utilised by amy at all (FIG. 16), but that anterograde transport is also affected.

FIG. 20 shows the dosage dependent effect of the three effector molecules on the transport of amy and amy-HDEL. The two GTPase mutants inhibit amy secretion strongly but have only weak inhibitory effect on the secretion of amy-HDEL, and thus confer a BFA-like effect. In contrast, Sec12p overproduction inhibited amy-HDEL secretion at least as much or even more than amy secretion, very much unlike the effect of BFA. These results suggest that Sec12p overproduction inhibits mainly the anterograde transport route (FIG. 16, route 1) without affecting the capacity for HDEL-receptor mediated retrieval from the Golgi (FIG. 16, route 2). In contrast, GTPase mutants of Sar1p and ARF1p inhibit both ER export and retrieval from the Golgi, but are indistinguishable from each other and both appear to exhibit a BFA-like effect.

Interference with ARF1 Dependent Transport Causes Mis-Sorting of a Vascular Protein

The difficulty to separate inhibition of COPI transport from COPII transport, as exemplified by the drug BFA (FIGS. 2 and 3) or the GTPase mutants of Sar1p and ARF1p (FIG. 20) is either due to the strong link between COPI and COPII transport, or due to multiple roles of ARF1p in Golgi derived transport. Inhibition of amy secretion by BFA or ARF1(Q71L)p could also occur distal to the cis-Golgi and represent the intra-Golgi forward transport (FIG. 16, route 3) that has often been attributed to COPI transport (Pelham and Rothman, 2000). To distinguish between these possibilities, we decided to subject a third soluble cargo molecule to the inhibition assays. Barley phytepsin was shown to leave the ER in a COPII dependent manner as α-amylase (Törmäkangas et al., 2001), but then leaves the Golgi to reach the vacuolar compartments of the plant cell (FIG. 16, route 5). The vacuolar form of phytepsin can be easily distinguished from its precursor in transit through the ER and the Golgi via the cleavage of the N-terminal propeptide. Moreover, saturation of vacuolar transport leads to mis-sorting to the cell surface, as seen for amy-HDEL, and under such conditions the unprocessed precursor is detected in both the cells and the culture medium.

FIG. 21 shows that overproduction of Sec12p leads to an accumulation of unprocessed precursor in the cells and an inhibition of phytepsin secretion. Transport to the vacuole is less affected, as seen by a maintained signal of the processed vacuolar form unless high concentrations of Sec12p encoding plasmids were co-transfected. Co-expression of Sar1(H74L)p leads to a similar effect on secretion but a more noticeable inhibition of vacuolar sorting as estimated by the diminishing signal of the processed form. In sharp contrast, co-expression of ARF1(Q71L)p causes a transient increase in the secretion of phytepsin, while transport to the vacuole is inhibited and the precursor accumulates in the cells. This increase in phytepsin secretion is observed at concentrations of the GTPase mutant at which α-amylase secretion is significantly inhibited (compare with FIG. 20). However, at higher concentrations of ARF1(Q71L)p, this secretion is abolished, particularly when using plasmid concentrations as in FIG. 19 (data not shown). This illustrates that without dosage experiments, the induced secretion of phytepsin might have gone unnoticed.

These results suggest that ARF1(Q71L)p mediated inhibition of amy secretion can not be due to a general inhibition of anterograde intra-Golgi transport and from the cis-Golgi to the cell surface (FIG. 16, routes 3 and 7). The data can only be explained if transport to the vacuole (route 5) is more sensitive to ARF1(Q71L)p than the anterograde transport from the ER via the Golgi to the plasma membrane (routes 1, 3 and 7), thus allowing faster secretion of phytepsin while secretion of amy is reduced.

BFA does not Mimic the Effect of ARF1(Q71L)

The data on the influence of ARF1(Q71L)p on the transport of amy and amy-HDEL suggested that the GTPase mutant exhibited a BFA-like effect (FIG. 20). To test if this similarity is also reflected on the sorting of phytepsin, we tested if BFA can induce secretion of this cargo molecule. Using the same experimental conditions as in FIG. 17, we show that BFA inhibits processing of the phytepsin precursor (FIG. 22). However, unlike the effect of ARF1(Q71L)p it does not cause an increase of phytepsin precursor secretion. Instead, secretion was inhibited at the lowest concentration of BFA used. This suggests that ARF1(Q71L)p interferes with ARF1p mediated transport in a completely different fashion from BFA. This is not surprising as BFA is expected to inhibit COPI vesicle formation itself (Chardin and McCormick, 1999; Peyroche et al., 1999), whereas a GTP restricted mutant of ARF1 does not (Nickel et al., 1998; Pepperkok et al., 2000).

ARF1(Q71L) Induced Secretion Occurs Through, Specific Inhibition of Transport to the Lytic Vacuole.

Plants contain more than one vacuolar compartment which are reached via different pathways dependent on a variety of sorting signals (Hoh et al., 1995; Paris et al., 1996; Neuhaus and Rogers, 1998). Although it has been shown that barley phytepsin is detected in both lytic and storage vacuoles (Paris et al., 1996), it is still unclear which type of vacuolar sorting signal is utilised by phytepsin (Törmäkangas et al., 2001). For this reason, we wanted to test two well established vacuolar sorting signals which are transplantable and can re-direct cargo molecules from the default pathway to vacuolar compartments.

We fused the C-terminal processed fragment of barley lectin (Bednarek and Raikhel, 1991) and the N-terminal propeptide of sweet potato sporamin (Koide et al., 1997) to the C-terminus of α-amylase, which was previously shown to maintain enzymatic activity upon fusion of small peptides (Crofts et al., 1999). The sporamin propeptide was shown to act as a sequence specific vacuolar sorting signal regardless of its location within the protein (Koide et al., 1997) and was predicted to act properly at the C-terminus of α-amylase. In contrast, the barley lectin propeptide has no sequence consensus but must be localised strictly at the C-terminus (Dombrowski et al., 1993). Both sorting signals are expected to target proteins to the same vacuole in tobacco (Schroeder et al., 1993), albeit via different pathways (Matsuoka et al., 1995). The amy fusion with the sporamin propeptide (amy-spo) would be predicted to act as a BP80 ligand (Koide et al., 1997; Matsuoka and Nakamura, 1999), whereas the fusion with the barley lectin propeptide (amy-bl) would be transported in a BP80-independent fashion (Matsuoka et al., 1995).

FIG. 23 shows that both C-terminal fusions (amy-spo and amy-bl) are retained in the cells compared to amylase, but that the fusion with the barley lectin propeptide exhibits more leakage to the medium than the sporamin propeptide fusion. More detailed analysis revealed that the two fusion proteins with wild type propeptides are mainly recovered from the supernatant of osmotically shocked cells, and also co-purify with the lytic vacuolar marker α-mannosidase, indicating that they reside predominantly in a vacuolar compartment rather than the ER/Golgi (data not shown).

We could also demonstrate that all fusion proteins exhibited equal specific enzymatic activities, as predicted from previous studies (Crofts et al., 1999).

Based on the detailed mutational analysis reported for the NPIRL motif illustrating the crucial role of the large alkyl side-chains of isoleucine and leucine (Matsuoka and Nakamura, 1999), we replaced these two residues by glycine (amy-spoM), yielding NPGRG. FIG. 23 illustrates that intracellular retention is completely abolished by this modification and results in normal secretion as seen for unmodified amy. This illustrates the sequence specific nature of this type of sorting signal (Neuhaus and Rogers, 1998). Addition of double glycines to the C-terminus of the barley lectin propeptide are known to render this signal non-functional (Dombrowski et al., 1993), and this is also confirmed for the amylase fusion (amy-blGG).

We subsequently tested the behaviour of these two fusion proteins in response to co-expression of ARF1(Q71L)p. FIG. 24A shows that the secretion of amy-spo was stimulated by ARF1(Q71L)p as observed for phytepsin, except that higher concentrations of the effector were needed in comparison with phytepsin (compare with FIG. 21). In contrast, ARF1(Q71L)p continuously inhibits secretion of amy-bl with increased dosage, as observed for amy and amy HDEL before. FIG. 24B shows the calculated secretion index which illustrates even better the completely opposite behaviour of the two cargo molecules. Also the total activity is affected in a differential fashion. Whereas total amy-spo levels increase slightly, amy-bl levels drop significantly with increasing ARF1(Q71L)p levels. The latter has also been observed for amy and amy-HDEL (data from FIG. 20, not shown).

To test if sequence specific vacuolar sorting per se is required to exhibit the ARF1(Q71L)p-induced secretion, we tested the mutagenised form of amy-spo containing the mutant motif NPGRG (amy-spoM). FIG. 25A shows that secretion is no longer stimulated but in fact inhibited by the ARF1 mutant, as seen for amy and amy-bl before. This confirms that induced secretion by the ARF1 mutant is a signature of sequence specific sorting signals, and thus BP80 ligands. This represents a novel tool to distinguish two different types of soluble cargo molecules following different sorting pathways.

To confirm results from FIG. 22 on phytepsin, showing that BFA does not exhibit an ARF1(Q71L)p-like effect, we tested the influence of increasing concentrations of BFA on the transport of amy-spo as well. FIG. 25B clearly shows that BFA does not induce secretion of the cargo molecule. Since phytepsin and amy-spo behave in a similar fashion, they are expected to follow similar transport routes, which suggests that at least a portion of phytepsin is transported by BP80. In contrast to BFA, ARF1(Q71L)p offers a unique opportunity to investigate BP80-mediated vacuolar sorting via a positive assay, the induced secretion of BP80-ligands. It also offers an excellent method to re-direct proteins, normally destined to the lytic vacuole, from the Golgi apparatus to the cell surface, leading to secretion. Secretion to the culture medium will constitute an easier production method due to the simple purification technique, and at the same time prevent proteolysis in the lytic vacuole. Such manipulation, which is only possible with the GTP-restricted mutant of ARF1, would thus constitute a major approach to increase productivity and cost effectiveness of heterologous protein expression by the secretory pathway.

### REFERENCES

Balch, W. E., McCaffery, J. M., Plutner, H., and Farquhar, M. G. (1994). Vesicular stomatitis virus glycoprotein is sorted and concentrated during export from the endoplasmic reticulum. Cell 76, 841-852.
Barlowe, C., and Schekman, R. (1993). SEC12 encodes a guanine-nucleotide-exchange factor essential for transport vesicle budding from the ER. Nature 365, 347-349.
Barlowe, C., d'Enfert, C., and Schekman, R. (1993). Purification and characterization of SAR1p, a small GTP-binding protein required for transport vesicle formation from the endoplasmic reticulum. J. Biol. Chem. 268, 873-879.
Barlowe, C., Orci, L., Yeung, T., Hosobuchi, A, Hamamoto, S., Salama, N., Rexach, M. F., Ravazzola, M., Amherdt, M., and Schekman, R. (1994). COPII: a membrane coat formed by Sec proteins that drive vesicle budding from the endoplasmic reticulum. Cell 77, 895-907.
Bar-Peled, M., and Raikhel, N. V. (1997). Characterization of AtSEC12 and AtSAR1. Proteins likely involved in endoplasmic reticulum and Golgi transport. Plant Phys. 114, 315-324.
Bednarek, S. Y., and Raikhel, N. V. (1991). The barley lectin carboxyl-terminal propeptide is a vacuolar protein sorting determinant in plants. Plant Cell 3, 1195-1206.
Cao, X., Rogers, S. W., Butler, J., Beevers, L., and Rogers, J. C. (2000). Structural requirements for ligand binding by a probable plant vacuolar sorting receptor. Plant Cell 12, 493-506.
Casadaban, M. J., and Cohen, S. N. (1980). Analysis of gene control signals by DNA fusion and cloning in Escherichia coli. J. Mol. Biol. 138, 179-207.
Chardin, P., and McCormick, F. (1999). Brefeldin A: the advantage of being uncompetitive. Cell 97, 153-155.
Crofts, A. J., Leborgne-Castel, N., Pesca, M., Vitale, A, and Denecke, J. (1998). BiP and calreticulin form an abundant complex that is independent of endoplasmic reticulum stress. Plant Cell 10, 813-824.
Crofts, A. J., Leborgne-Castel, N., Hillmer, S., Robinson, D. G., Phillipson, B., Carlsson, L. E., Ashford, D. A., and Denecke, J. (1999). Saturation of the endoplasmic reticulum retention machinery reveals anterograde bulk flow. Plant Cell 11, 2233-2248.
Denecke, J., and Vitale, A. (1995). The use of protoplasts to study protein synthesis and transport by the plant endomembrane system. Methods Cell Biol. 50, 335-348.
Denecke, J., Botterman, J., and Deblaere, R. (1990). Protein secretion in plant cells can occur via a default pathway. Plant Cell 2, 51-59.
Denecke, J., De Rycke, R., and Botterman, J. (1992). Plant and mammalian sorting signals for protein retention in the endoplasmic reticulum contain a conserved epitope. EMBO J. 11, 2345-2355.
Denecke, J., Carlsson, L. E., Vidal, S., Hoglund, A. S., Ek, B., van Zeijl, M. J., Sinjorgo, K. M., and Palva, E. T. (1995). The tobacco homolog of mammalian calreticulin is present in protein complexes in vivo. Plant Cell 7, 391-406.
d'Enfert, C., Gensse, M., and Gaillardin, C. (1992). Fission yeast and a plant have functional homologues of the Sar1 and Sec12 proteins involved in ER to Golgi traffic in budding yeast. EMBO J. 11, 4205-4211.
d'Enfert, C., Wuestehube, L. J., Lila, T., and Schekman, R. (1991a). Sec12p-dependent membrane binding of the small GTP-binding protein Sar1p promotes formation of transport vesicles from the ER. J. Cell Biol 114, 663-670.
d'Enfert, C., Barlowe, C., Nishikawa, S., Nakano, A., and Schekman, R. (1991b). Structural and functional dissection of a membrane glycoprotein required for vesicle budding from the endoplasmic reticulum. Mol. Cell Biol. 11, 5727-5734.
Dombrowski, J. E., Schroeder, M. R., Bednarek, S. Y., and Raikhel, N. V. (1993). Determination of the functional elements within the vacuolar targeting signal of barley lectin. Plant Cell 5, 587-596.
Hara-Nishimura, I., Shimada, T., Hatano, K., Takeuchi, Y., and Nishimura, M. (1998). Transport of storage proteins to protein storage vacuoles is mediated by large precursor-accumulating vesicles. Plant Cell 10, 325-836.
Hardwick, K. G., Boothroyd, J. C., Rudner, A. D., and Pelham, H. R. (1992). Genes that allow yeast cells to grow in the absence of the HDEL receptor. EMBO J. 11, 4187-4195.
Hoh, B., Hinz, G., Jeong, B. K., and Robinson, D. G. (1995). Protein storage vacuoles form de novo during pea cotyledon development. J. Cell Sci. 108 (Pt 1), 299-310.
Humair, D., Hernandez Felipe, D., Neuhaus, J. M., and Paris, N. (2001). Demonstration in yeast of the function of BP-80, a putative plant vacuolar sorting receptor. Plant Cell 13, 781-792.
Kirsch, T., Saalbach, G., Raikhel, N. V., and Beevers, L. (1996). Interaction of a potential vacuolar targeting receptor with amino- and carboxy-terminal targeting determinants. Plant Phys. 111, 469-474.
Kirsch, T., Paris, N., Butler, J. M., Beevers, L., and Rogers, J. C. (1994). Purification and initial characterization of a potential plant vacuolar targeting receptor. Proc. Natl. Acad. Sci. USA 91, 3403-3407.
Koide, Y., Hirano, H., Matsuoka, K., and Nakamura, K. (1997). The N-terminal propeptide of the precursor to sporamin acts as a vacuole-targeting signal even at the C terminus of the mature part in tobacco cells. Plant Phys. 114, 863-870.
Leborgne-Castel, N., Jelitto-Van Dooren, E. P., Crofts, A. J., and Denecke, J. (1999). Overexpression of BiP in tobacco alleviates endoplasmic reticulum stress. Plant Cell 11, 459-470,
Maliga, P., Sz-Breznovits, A., and Marton, L. (1973). Streptomycin-resistant plants from callus culture of haploid tobacco. Nat. New Biol. 244, 29-30.
Matsuoka, K, and Nakamura, K. (1999). Large alkyl side-chains of isoleucine and leucine in the NPIRL region constitute the core of the vacuolar sorting determinant of sporamin precursor. Plant Mol. Biol. 41, 825-835.
Matsuoka, K., Bassham, D. C., Raikhel, N. V., and Nakamura, K. (1995). Different sensitivity to wortmannin of two vacuolar sorting signals indicates the presence of distinct sorting machineries in tobacco cells. J. Cell Biol. 130, 1307-1318.
Murashige, R., and Skoog, F. (1962). A revised medium for rapid growth and bioassays with tobacco tissue cultures. Physiol. Plant. 15, 473-497.
Neuhaus, J. M., and Rogers, J. C. (1998). Sorting of proteins to vacuoles in plant cells. Plant Mol. Biol, 38, 127-144.
Nickel, W., Malsam, J., Gorgas, K., Ravazzola, M., Jenne, N., Helms, J. B., and Wieland, F. T. (1998). Uptake by COPI-coated vesicles of both anterograde and retrograde cargo is inhibited by GTPgammaS in vitro J. Cell Sci. 111 (Pt 20), 3081-3090.
Nishikawa, S., Hirata, A., and Nakano, A. (1994). Inhibition of endoplasmic reticulum (ER)-to-Golgi transport induces relocalization of binding protein (BiP) within the ER to form the BiP bodies. Mol. Biol. Cell 5, 1129-1143.
Nishimura, N., and Balch, W. E. (1997). A di-acidic signal required for selective export from the endoplasmic reticulum. Science 277, 556-558.
Paris, N., Stanley, C. M., Jones, R. L., and Rogers, J. C. (1996). Plant cells contain two functionally distinct vacuolar compartments. Cell 85, 563-572.
Paris, N., Rogers, S. W., Jiang, L., Kirsch, T., Beevers, L, Phillips, T. E., and Rogers, J. C. (1997). Molecular cloning and further characterization of a probable plant vacuolar sorting receptor. Plant Phys. 115, 29-39.
Pelham, H. R., and Rothman, J. E. (2000). The debate about transport in the Golgi—two sides of the same coin? Cell 102, 713-719.
Pepperkok, R., Whitney, J. A., Gomez, M., and Kreis, T. E. (2000). COPI vesicles accumulating in the presence of a GTP restricted arf1 mutant are depleted of anterograde and retrograde cargo. J. Cell Sci. 113 (Pt 1), 135-144.
Peyroche, A., Antonny, B., Robineau, S., Acker, J., Cherfils, J., and Jackson, C. L. (1999). Brefeldin A acts to stabilize an abortive ARF-GDP-Sec7 domain protein complex: involvement of specific residues of the Sec7 domain. Mol. Cell 3, 275-285.
Phillipson, B. A., Pimpl, P., Crofts, A. J., Taylor, J. P., Movafeghi, A., Robinson, D. G., and Denecke, J. (2001). Secretory bulk flow of soluble proteins is COPII dependent. Plant Cell 13, 2005-2020.
Pimpl, P., Movafeghi, A., Coughlan, S., Denecke, J., Hillmer, S., and Robinson, D. G. (2000). In Situ Localization and in Vitro Induction of Plant COPI-Coated Vesicles. Plant Cell 12, 2219-2236.
Saito, Y., Kimura, K., Oka, T., and Nakano, A. (1998). Activities of mutant Sar1 proteins in guanine nucleotide binding, GTP hydrolysis, and cell-free transport from the endoplasmic reticulum to the Golgi apparatus. J. Biochem. 124, 816-823.
Schroeder, M. R., Borkhsenious, O. N., Matsuoka, K., Nakamura, K., and Raikhel, N. V. (1993). Colocalization of barley lectin and sporamin in vacuoles of transgenic tobacco plants. Plant Phys. 101, 451-458.
Takeuchi, M., Tada, M., Saito, C., Yashiroda, H., and Nakano, A. (1998). Isolation of a tobacco cDNA encoding Sar1 GTPase and analysis of its dominant mutations in vesicular traffic using a yeast complementation system. Plant Cell Physiol. 39, 590-599.
Takeuchi, M., Ueda, T., Sato, K, Abe, H., Nagata, T., and Nakano, A. (2000) A dominant negative mutant of Sar1 GTPase inhibits protein transport from the endoplasmic reticulum to the Golgi apparatus in tobacco and Arabidopsis cultured cells. Plant J. 23, 517-525.
Törmäkangas, K., Hadlington, J. L., Pimpl, P., Hillmer, S., Brandizzi, F., Teeri, T. H., and Denecke, J. (2001). A vacuolar sorting domain may also influence the way in which proteins leave the endoplasmic reticulum. Plant Cell 13, 2021-2032.
Toyooka, K., Okamoto, T., and Minamikawa, T. (2000). Mass transport of proform of a KDEL-tailed cysteine proteinase (SH-EP) to protein storage vacuoles by endoplasmic reticulum-derived vesicle is involved in protein mobilization in germinating seeds. J. Cell Biol. 148, 453-463.
Vitale, A., and Denecke, J. (1999). The endoplasmic reticulum-gateway of the secretory pathway. Plant Cell 11, 615-628.
Wieland, F. T., Gleason, M. L., Serafini, T. A., and Rothman, J. E. (1987). The rate of bulk flow from the endoplasmic reticulum to the cell surfaces Cell 50, 289-300.

1. A method of increasing heterologous protein production in a cell by controlling proteolysis in the post endoplasmic reticulum (ER) compartment of the cell.

2. A method of increasing heterologous protein expression/production in a cell comprising limiting and/or decreasing proteolysis in said cell by:
(i) limiting/preventing export of proteins from the ER to the post ER compartment of the cell;
(ii) re-directing proteins from the vacuolar sorting route back to the ER; and/or
(iii) re-directing proteins from the vacuolar route on towards the cell surface.

3. A method according to claim 1, wherein the cell is of plant, fungal, yeast or mammalian origin.

4. A method according to claim 1, wherein the cell contains heterologous DNA encoding a mammalian serum protein.

5. A method according to claim 1, wherein the cell contains heterologous DNA encoding a mammalian encoding a mammalian hormone.

6. A method according to claim 1, wherein proteolysis is controlled by limiting/preventing export of proteins from the ER by inhibition of COPII transport.

7. A method according to claim 6 wherein inhibition of COPII transport is achieved by either of the following methods:
(i) inhibition of COPII dependent vesicle budding via overproduction of Sar1-specific guanosine exchange factor;
(ii) co-expression of mutant GTPase which is less sensitive to its GTPase activating protein and as a result is defective in GTP hydrolysis or;
(iii) co-expression of mutant GTPase which is less sensitive to its GTPase activating protein and as a result is defective in GTP hydrolysis.

8. A method according to claim 7 wherein the exchange factor is Sec12p or an isoform thereof.

9. A method according to claim 7 wherein the mutant GTPase is Sar1 or ARF1 or isoforms thereof.

10. A method according to claim 1, wherein re-directing proteins from the vacuolar sorting route back to the ER is by co-expression of a modified vacuolar sorting receptor which carries an engineered retention signal.

11. A method according to claim 10 wherein the receptor is BP80 or VPS10 or close isoforms thereof.

12. A method according to claim 10 wherein the cell is adapted so that the BP80 phenotype is depleted by producing a BP80-mycHDEL receptor.

13. A method according to claim 10 wherein the BP80 receptor is specifically engineered so as to reach a cell surface with its protein cargo, so that the protein is secreted at the cell surface.

14. A method according to claim 1, wherein cells containing heterologous DNA are incubated in appropriate fermentation/incubation media for a period to generate sufficient cell mass.

15. A method according to claim 1, wherein the proteolysis control step is conducted for about 12 to 48 hrs before harvesting the heterologous protein.

16. A method according to claim 1, for use in large scale production of heterologous proteins.

17. A method according to claim 1, further comprising any one or more of the following steps:
(i) harvesting the protein;
(ii) purification of the protein;
(iii) modification of the protein;
(iv) formulating the protein in a suitable diluent, carrier or excipient; or
(v) lyophilising the protein.

18. A method of producing heterologous mammalian proteins comprising:
(i) incubating cells containing heterologous DNA encoding a protein of choice in appropriate incubation media until sufficient cell mass is generated;
(ii) reducing export of proteins from the ER to the post ER compartment of the cell and/or re-directing proteins from the vacuolar sorting route back to the ER or on towards the cell surface and;
(iii) harvesting the heterologous protein.

19. A method according to claim 18 further including the step of disposal of cell debris by fermentation to methane and/or incineration of solid waste.

20. A method according to claim 18 further comprising any one or more of the following steps:
(i) harvesting the protein;
(ii) purification of the protein;
(iii) modification of the protein;
(iv) formulating the protein in a suitable diluent, carrier or excipient; or lyophilising the protein.

21. A method of producing heterologous mammalian serum proteins comprising:
(i) incubating cells containing heterologous DNA encoding the serum protein of choice in appropriate incubation media until sufficient cell mass is generated;
(ii) reducing export of proteins from the ER to the post ER compartment of the cell and/or re-directing proteins from the vacuolar sorting route back to the ER or on towards the cell surface and;
(iii) harvesting the heterologous serum protein.

22. A method according to claim 21 further including the step of disposal of cell debris by fermentation to methane and/or incineration of solid waste.

23. A method according to claim 21, wherein proteolysis is controlled by limiting/preventing export of proteins from the ER by inhibition of COPII transport.

24. A heterologous protein product produced by the method according claim 18.

25. A cell adapted so that proteolysis is limited and/or decreased.

26. A cell adapted by the method of claim 1 so that proteolysis is limited or decreased.

27. The method according to claim 26 comprising increasing production of a heterologous protein.

28. A method according to claim 2, wherein the cell is of plant, fungal, yeast or mammalian origin.

29. A method according to claim 2, wherein the cell contains heterologous DNA encoding a mammalian serum protein.

30. A method according to claim 2, wherein the cell contains heterologous DNA encoding a mammalian encoding a mammalian hormone.

31. A method according to claim 2, wherein proteolysis is controlled by limiting/preventing export of proteins from the ER by inhibition of COPII transport.

32. A method according to claim 31 wherein inhibition of COPII transport is achieved by either of the following methods:
(iv) inhibition of COPII dependent vesicle budding via overproduction of Sar1-specific guanosine exchange factor;
(v) co-expression of mutant GTPase which is less sensitive to its GTPase activating protein and as a result is defective in GTP hydrolysis or;
(vi) co-expression of mutant GTPase which is less sensitive to its GTPase activating protein and as a result is defective in GTP hydrolysis.

33. A method according to claim 32 wherein the exchange factor is Sec12p or an isoform thereof.

34. A method according to claim 32 wherein the mutant GTPase is Sar1 or ARF1 or isoforms thereof.

35. A method according to claim 2, wherein re-directing proteins from the vacuolar sorting route back to the ER is by co-expression of a modified vacuolar sorting receptor which carries an engineered retention signal.

36. A method according to claim 35 wherein the receptor is BP80 or VPS10 or close isoforms thereof.

37. A method according to claim 35, wherein the cell is adapted so that the BP80 phenotype is depleted by producing a BP80-mycHDEL receptor.

38. A method according to claim 35, wherein the BP80 receptor is specifically engineered so as to reach a cell surface with its protein cargo, so that the protein is secreted at the cell surface.

39. A method according to claim 2, wherein cells containing heterologous DNA are incubated in appropriate fermentation/incubation media for for a period to generate sufficient cell mass.

40. A method according to claim 2, wherein the proteolysis control step is conducted for about 12 to 48 hrs before harvesting the heterologous protein.

41. A method according to claim 2, for use in large scale production of heterologous proteins.

42. A method according to claim 2, further comprising any one or more of the following steps:
(i) harvesting the protein;
(ii) purification of the protein;
(iii) modification of the protein;
(iv) formulating the protein in a suitable diluent, carrier or excipient; or
(v) lyophilising the protein.

43. A heterologous protein product produced by the method according to claim 21.

44. A cell comprising heterologous nucleic acids so that proteolysis is limited and/or decreased.

45. The cell of claim 44, wherein said heterologous nucleic acids encode a serum protein or a hormone.

46. A cell adapted by the method of claim 2 so that proteolysis is limited or decreased.

47. The method according to claim 46 comprising increasing production of a heterologous protein.
